SEQUENCE LISTING (2 sequences)

SEQ ID NO: 1 - protein, 42 aa, Mus musculus
DAEFRHDSGFEVRHQKLVFFAEDVGSNKGAIIGLMVGGVVIA

SEQ ID NO: 2 - protein, 42 aa, Homo Sapiens [sequence omitted]

(19) United States
(12) Patent Application Publication
(10) Pub. No.: US 2010/0105098 A1
(43) Pub. Date: Apr. 29, 2010

(54) Methods of Identifying Disease Biomarkers in the Lense of the Eye

(76) Inventors: Peter Frederiske, Maplewood, NJ (US); Gordon Thomas, Princeton, NJ (US)

Correspondence Address: KLAUBER & JACKSON, 411 HACKENSACK AVENUE, HACKENSACK, NJ, 07601, US

(21) Appl. No.: 12/308,974

(22) PCT Filed: Jun. 9, 2007
(86) PCT No.: PCT/US2007/015156

§ 371 Date: Oct. 20, 2009

Related U.S. Application Data
(60) Provisional application No. 60/817,615, filed on Jun. 29, 2006.

(51) Int. Cl. C12Q 1/02

(52) U.S. Cl. 435/29

(57) ABSTRACT

The present invention relates to methods for the early diagnosing of an amyloid-related disorder or a predisposition thereto in a subject through the detection or monitoring of a metal-protein complex in the ocular lens, wherein said metal-protein complex comprises at least one amyloid protein.

### CROSS-REFERENCE TO RELATED APPLICATIONS

Under 35 U.S.C. §119(e) this application claims the benefit of U.S. Provisional Application No. 60/817,615 filed Jun. 29, 2006, which is hereby incorporated by reference in its entirety.

### STATEMENT REGARDING FEDERALLY SPONSORED RESEARCH

The U.S. Government has certain rights in this invention pursuant to Grant No.: EY12377 (Apr. 1, 2000-Mar. 31, 2003) to Peter H. Frederikse, awarded by the National Institutes of Health (NIB), National Eye Institute (NEI).

### INCORPORATION BY REFERENCE

In compliance with 37 C.F.R. §1.52(e)(5), the sequence information contained on compact disc, file name: Frederikse SEQ List_ST25.txt; size 2 KB; created on: Jun. 28, 2007; using PatentIn-3.4, and Checker 4.4.0 is hereby incorporated by reference in its entirety. The Sequence Listing information recorded in computer readable form (CRF) is identical to the written Sequence Listing provided herewith. The data in the paper copy of the Sequence Listing, and Computer Readable Form of the Sequence Listing submitted herewith contain no new matter, and are fully supported by the priority application, U.S. Provisional Patent Application No. 60/817,615.

### FIELD OF THE INVENTION

This invention relates to methods of detecting, diagnosing, prognosing, staging, and/or monitoring of pathological conditions through the identification of one or more biomarkers in the lens of the eye of a subject.

### BACKGROUND

β-amyloid peptide (Aβ) aggregates are hallmarks of Alzheimer's Disease (AD) pathology in brain. However, fibrils and plaques are characteristic of later disease stages. Aβ peptides are derived from the β-amyloid precursor protein (APP or AβPP) located on chromosome 21 and are associated with early-onset AD, and early-onset cataract in Down syndrome. AβPP is alternatively spliced to produce a 695-770 amino acid trans-membrane glycoprotein located in intracellular cargo vesicle membranes and at the cell surface. In contrast to brain where the 695aa form predominates, longer splice forms are the major form in lens, and evidence suggests longer APP proteins may have greater deleterious potential. APP is cleaved by γ-secretase (presenilin) and β-secretase proteases to release 39-43 amino acid Aβ peptide (˜4 kDa). Alternatively, α-secretase can cleave APP within the Aβ peptide region that lies partially within the trans-membrane domain in a non-amyloidogenic pathway. Aβ1-40 amino acid form (FIG. 1) is the predominant form in brain and lens, however human Aβ1-42 is more closely associated with AD pathology in large part due to much higher Cu affinity. Although human Aβ1-40 has lower Cu affinity, it was shown in vitro that Aβ1-40 peptides produce ˜50% of the H₂O₂ that is produced by Aβ1-42 peptides.

Aβ pathology has been demonstrated in human cataract and in several cataract animal models, and conversely, specific cataract formation has been demonstrated in human AD donor lenses and in transgenic AD mouse models. This has led to an important understanding that AD animal models provide fundamentally germane models of age-dependent cataract formation, and build on generalized stress models of senile cataract that have been predominant in cataract research. In addition, these studies support the conclusion that lens and brain extensively share the same biological diathesis model for age-dependent disease, and that Aβ production in lens is a primary and consistent diagnostic biomarker that can be used for the diagnosis of senile cataract. Moreover, lens Aβ pathology also provides a critical biomarker of Aβ pathology in brain for diagnosis of AD in human patients.

Aβ aggregation is determined in large part by metal interactions, particularly with Cu and Zn. Aβ peptides have a very high affinity for Cu, and Cu readily displaces Zn in Aβ complexes. However, Cu and Zn can produce different complexes with Aβ. Zn only contributes to Aβ aggregation and fibril formation, whereas Cu promotes fibril formation, but also can form small soluble redox active Aβ-Cu complexes that oxidize cellular component substrates and produce hydrogen peroxide (H₂O₂).

Currently, there are no means to monitor, non-invasively, Aβ-metal complexes in the lens of the eye for purposes of detecting, diagnosing, prognosing or staging cataracts and/or AD pathology in the brain.

### SUMMARY OF THE INVENTION

The present invention relates to the discovery that the detection and measurement of Aβ-metal conjugates or complexes in the ocular lens of a subject are excellent biomarkers of amyloid-related disease progression, and can be monitored through the use of non-invasive Raman spectroscopic techniques. Raman spectroscopy identifies Aβ-metal complexes at the molecular level in situ. An important advantage of the instant methods includes the ability to detect small soluble Aβ forms in the transparent lens that appear before significant light scattering is detected and before detection of significant higher order amyloid 2° protein structures is possible. Thus, the present methods provide for earlier detection and diagnosis of amyloid-related pathologies. The identification of small soluble AB-metal complexes with Raman spectroscopy has significant potential to provide insights into disease mechanisms and provide diagnostic information about early diseases stages in lens and brain.

Therefore, in certain aspects the methods described herein encompass the identification and measurement of small soluble Aβ-metal complexes with Raman spectroscopy for the detection, diagnosis, prognosis and/or staging of diseases in the ocular lens of a subject. Increases in Aβ biomarkers are a key element in the hallmark accumulation of Copper (Cu) in cataract, and therefore, the detection or measurement of Aβ-metal complexes in the ocular lens of a subject can be used for early detection or to determine predisposition and/or progression of eye diseases, for example, age-related or AD-associated cataracts.

The basic mechanisms of Alzheimer disease (AD) pathology are fundamentally involved in cataract. Evidence indicates that the ocular lens and brain share the same biological diathesis model for age-dependent disease. It has been shown that Aβ production in the ocular lens is a primary and consistent diagnostic biomarker that can be used for the diagnosis of Aβ pathology in brain for diagnosis of AD in a subject. Therefore, in an additional aspect, the instant disclosure encompasses methods for the identification and measurement of small soluble Aβ-metal complexes in the ocular lens of subject through the use of Raman spectroscopic techniques for the early detection, diagnosis, prognosis and/or staging of AD in a subject.

Additional objects and advantages of the present invention will be appreciated by one of ordinary skill in the art in light of the instant claims, description, and examples. These additional objects and advantages are expressly included within the scope of the present invention.

### BRIEF DESCRIPTION OF THE DRAWINGS

FIG. 1: Processing of β-amyloid precursor protein by β-secretase and γ-secretase.

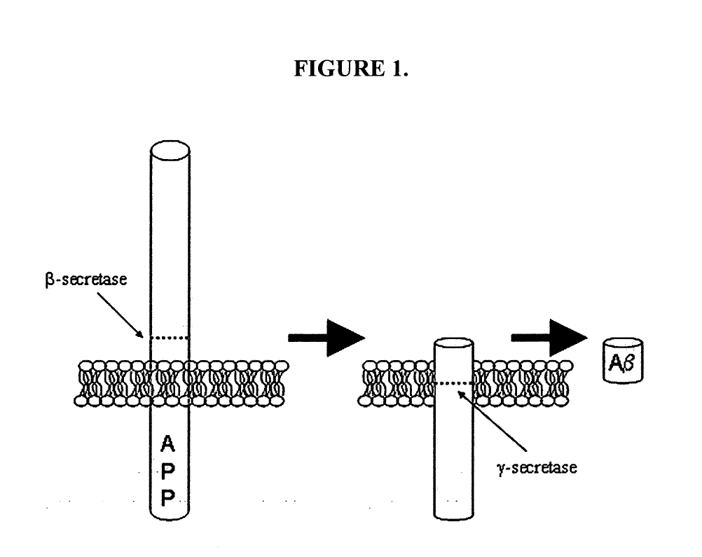

FIG. 2: Mammalian Alzheimer β-amyloid peptide sequences showing His & Tyr residues key in metal binding (indicated in bold).

FIG. 3: Histidine amino acid imidazole nitrogens interactions with Zn and Cu. Cu interacts in a pH dependent manner. N-tau binding is linked with fibrils and aggregation. N-pi interactions with intra-molecular & redox competent forms.

FIG. 4: Models of N-tau binding of Cu or Zn leads to inter-Aβ binding and fibrils. N-pi binding of Cu only leads to intra-molecular binding, Aβ-Cu monomers dimers acquire Super Oxide Dismutase (SOD) activity.

FIG. 5: Aβ-metal aggregation determined by sedimentation confirms Zn binding leads to inter-Aβ binding; and Cu binding is pH specific: monomer formation at 7.4 & aggregates at low pH.

FIG. 6: A) Aβ in Human cataract lens. B) Marked drop-off in pH from pH 7.4 to pH 6.5-6.8 occurs near the lens perimeter and consistent with supranuclear cataract production. C) Supranuclear cataract formation in Tg2576 and D) Human AD lens controls similar to rabbit lens in FIG. 21.

FIG. 7: In vitro analysis of Aβ peptides interaction with Cu and Zn metal atoms, analyzed with Raman Spectroscopy. The Spectra from 1500 to 1770 cm-1 is shown. Amide I peak intensity at ˜1670 cm-1 indicates β-sheet formation consistent with amyloid protein structure and Aβ fibril formation and aggregation as modeled in Panel E. Signature intensity at 1604 cm-1 identifies N-tau metal binding consistent with aggregation, β-amyloid, and fibrils, Amide I and N-tau signature intensities are relatively decreased in Panel B and indicate intra-molecular Aβ-Cu binding at physiological pH and modeled in Panel E. Panel D demonstrates that the identical signature bands are detected in Raman spectra using senile plaque core material from human AD donor brain tissue. These data demonstrate Raman spectroscopy can be used to identify & characterize specific Aβ-metal complexes in vitro & in brain tissue pathology.

FIG. 8: YAC hAβPP Tg mouse lenses produce cargo vesicle trafficking defects similar to neurons. PANEL A: YAC hAβPP mouse lens with cataractous lens fibers and dense deposits. PANEL B: hAβPP mouse lens fibers are disorganized with many membrane bound vesicles, and are also characteristic in human senile cataract (PANEL C Top; ref. 1). Likewise, transgenic AβPP or Kinesin expression in neurons disrupts cargo vesicle traffic with increased vesicles (PANEL C Lower; 51,118) and lateral boutons (PANEL D; 122). These observations led studies us to demonstrate a strikingly level of shared AβPP/kinesin based cargo & synaptic vesicle transport machinery, neuron specific synapsins and synaptic vesicle proteins in lens fiber cells, during early embryonic and adult development (see FIG. 9). hAβPP single copy Tg mice carry a 400 kb fragment from human chromosome 21 with all introns/exons, normal gene-splicing, and driven by native promoter elements. This model was examined first as hAβPP mice recapitulates native physiological AβPP expression patterns, native regulatory elements respond normally to systemic physiology, and can better obviate gratuitous tissue-preferred over-expression effects. (please see appendix for high quality photographs)

FIG. 9: In late disease, Aβ detected in hAβPP lenses co-localizes with deposits containing crystallin proteins in swollen fiber cells. For contrast, Aquaporin 0 (MIP26) in membranes is not localized to deposits. At right, Congo Red identifies plaques in swollen regions. Note IHC and amyloid dye reagents do not fully penetrate dense plaques,

FIG. 10: Overlapping distributions of AβPP, JIP1b and Kinesin motor proteins, Tau, and “neuron-specific” synaptic vesicle proteins and Synapsins occur along the axial length of fetal lens fiber cells (and in adult; not shown). In neurons these proteins link cargo and synaptic vesicles to microtubules for transport to distal cell surfaces. Key gene regulation is also shared with neuron differentiation: Synapsin II & III, but not Synapsin I are expressed early during rapid E17 fiber cell elongation. B. Later, RT-PCR shows post-natal lens fiber maturation utilizes Synapsins I & II, but not Synapsin III similar to neurons in vitro or in vivo. C. Key phospho-regulation of Synapsin at Ser9 (site-1) also occurs in lens. In neurons, Ser-9 phosphorylation governs Synapsin-coated vesicle cytoskeleton release, Synapsin mediated actin polymerization, & Synapsin regulated neuron cell differentiation. Panel C (A-F) shows anti-Site-1 Phospho- and Dephospho-Synapsin antibodies identify discrete and differential distributions of Synapsin proteins in rapidly elongating E17 lens fiber cells, consistent with vesicle release at the apical tips of neurons. D. E.M photos by Lo demonstrate neuron-like microtubules (Arrows) and vesicles (Black Arrows) in lens fibers, with end-on pin-wheels indicating neuron-like axial polarity by Lo et al.

FIG. 11: Tg2576 mice produce significantly more cataracts with greater severity by 3-400 days vs. ˜20 mos in wt.

FIG. 12: We note comparison of mouse and human AβPP to produce behavioral deficits Hsiao noted similar but delayed deficits in MoAβPP Tg mouse lenses vs. HuAβPP.

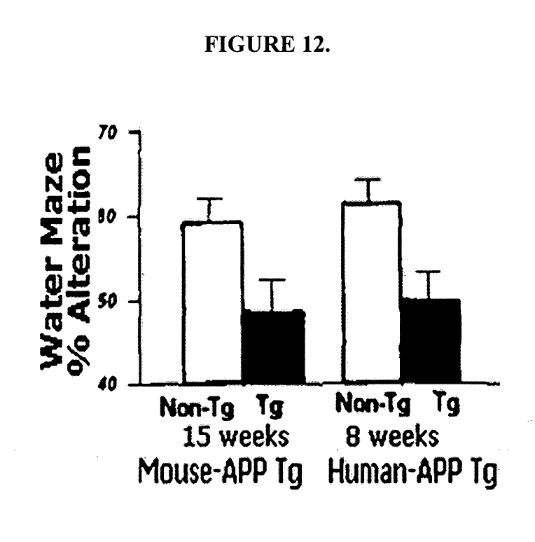

FIG. 13: Partial compilation of Atomic Absorption Spectrometry data over 20 yrs. for Cu accumulation in lens with aging, and more so in cataract formation, in species expressing high Cu affinity Aβ (e.g. not rat).

FIG. 14: Overlapping distribution of increased Aβ (B vs. E) and Cu (C vs. F) in congenital cataract guinea pig lens (panels D-F) vs. wt (panels A-C). For Cu stain, HCl Zn/Fe pre-leaching had no effect (not shown). Rt. Top: Aβ increases in cataractous mutant lens. Below, RT-PCR of AβPP exons 6-9 in mutant vs. wt lens using equal total RNA shows predominant AβPP transcripts are elevated relative to total RNA, These data provide a further example of the fundamental role of AβPP & Aβ in lens pathology.

FIG. 15: Thiamine deprived mice produce local accumulation of Aβ in regions of lens fiber degeneration: A) Lens H'nE stain. B) Fiber cell degradation w/locally increased Aβ.

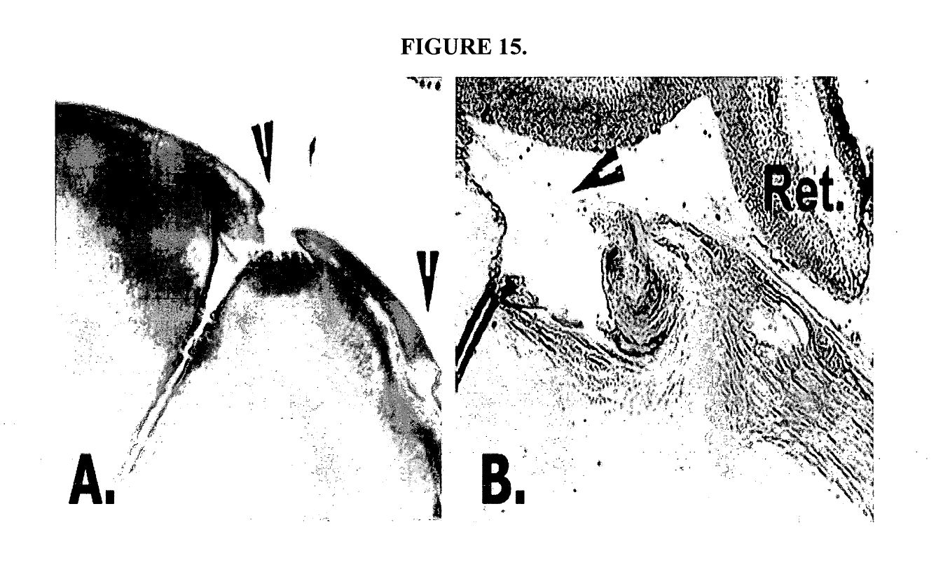

FIG. 16: Raman System and Data Collection Procedure. The apparatus that is used to take measurements consists of a CCD detector coupled to a spectrometer with a digital output to a computer, a diode laser, and an excitation/collection Raman probe. In a preferred embodiment, the system includes an 830 nm diode laser with an adjustable power output, maximum of 270 mW, a 5 cm-1 resolution spectrometer, with an electronically cooled CCD row detector, and an Inphotonics™ Raman probe. Unlike other CCD detectors using expensive and large liquid nitrogen cooling systems, the CCD in this embodiment incorporates an electronically cooled detector to reduce noise. The 830 nm NIR laser source is chosen for the system to diminish extra fluorescence background and to minimize the tissue damage, however wavelength and power (mW) may also be modified to increase the Aβ-Cu signal-to-noise ratio.

FIG. 17: Immunoblot detection of ˜4 kDa Aβ peptides using 4G8 monoclonal antibody. Total Rabbit lens proteins resolved on Tricine gels and blotted to filters were probed with antibodies and visualized by HRP detection. Below, are densitometry measurements of bands in the gel above. Image J was used to score bands relative to adjacent protein-free regions.

FIG. 18: ELISA analysis of four high cholesterol/Cu lenses and four control diet rabbit lenses showing Aβ increases.

FIG. 19: Immunoblot analysis of AβPP shows slight if any increase in cataracts in cholesterol copper fed rabbit lenses.

FIG. 20: Hallmark Cu accumulation in cholesterol/Cu rabbit lenses vs. control lenses. Two assays each w/2 lenses in each group.

FIG. 21: Cataracts present in high cholesterol/Cu fed rabbits lenses (right) compared to clear controls (left) Cataracts are also consistent with Aβ pathology, increased Aβ, elevated Cu, and detection of Aβ-metal complexes by Raman spectroscopy.

FIG. 22: Three Raman spectra traces graphed above each other. Top: Raman spectra of an intact lens from a high cholesterol/Cu rabbit. Middle: Spectra from a lens from normal diet rabbit. The bottom trace is the computed ‘difference’ curve showing changes between the two spectra vs. a constant reference. Amide I (˜1665 cm⁻¹) identifies strong lens β-sheet, content consistent with numerous previous Raman lens studies (e.g. 104). Signature changes in signals for N-tau Aβ-Cu or Zn interaction are seen at ˜1602 cm⁻¹ and agree with increase observed in biochemical assays measuring elevated Aβ and elevated Cu in experimental rabbit lenses, and consistent with Cu as a hallmark cataract biomarker. Little N-pi signal is detected at 1595 cm⁻¹ suggesting Aβ-Cu or Zn complexes predominantly accrue in lenses, that involve N-tau imidazole His interaction and indicates much Aβ-metal is involved in fibrillar or aggregated forms.

FIG. 23: Left panels: Raman spectra showing the region from 1500-1700 cm⁻¹ from lenses of cholesterol/Cu fed rabbits and just below from normal diet control rabbit lenses. Spectra are paired randomly and the computed difference curves are shown in bottom tracings. Remarkably, spectra from cholesterol/Cu fed rabbits produce increased signature Raman scattering intensities at 1604 cm⁻¹ not present in control lenses. This signal is diagnostic of Aβ-metal complex increases corresponding with lens opacification (FIG. 20) and with increases in Aβ and Cu. These data indicate lens Aβ pathology corresponds with production of significant AD pathology in brain in this rabbit model. The Raman spectra shown in the panels on the right show senile plaque core material from AD brain tissue. We also note Increased Cu measured in our pilot study also agrees with atomic absorption spectrometry studies of biomarker lens Cu accumulation.

FIG. 24: Western blot detection of Aβ and AβPP in intact Monkey lenses exposed to oxidative stress agents.

FIG. 25: EMSA Gel-shift assay of AP-1 activation in the same monkey lens samples as above that were exposed to stress.

FIG. 26: Cultured lens exposed to a bolus of 250 μM H₂O₂ incubated for 24 hrs. Light scattering indicative of lens opacification increases over time.

### DETAILED DESCRIPTION OF THE INVENTION

Aβ pathology has a key role in lens disease, the accumulation of Cu in cataract, and underlies its relationship with associated changes in brain. Key Aβ secretases and proteases are also present in lens regions overlapping with readily detectable Aβ in the lens. To date, Aβ deposits have been demonstrated in late stage lens disease occurring in mature human senile cataract, and in cataract linked with AD.

The present invention relates to the discover that small soluble Aβ-metal complexes are produced early-on in the amyloid-related disease process and exist as monomers or small soluble oligomers. However, unlike amyloid protein deposition in brain tissue which is primarily extracellular, ocular deposition in lens cortical fiber cells is cytosolic. Evidence indicates that monomers or perhaps also Aβ-metal dimers acquire redox competent Super Oxide Dismutase (SOD)-like enzymatic activity that produces H₂O₂ to create oxidative stress, which disrupts cell function and contributes to cell death. Thus, small non-aggregated Aft, and Aft-metal forms appear early in the disease, appear to be more toxic, and serve as a convenient means for early detection, diagnosis, and monitoring of amyloid-related diseases.

As used herein, an “amyloid-related” disorder is one that is marked by the accumulation, deposition, collection or agglomeration of an Aβ-metal complex or fragment thereof in the ocular lens or brain of an individual. Amyloid-related disorders contemplated as being within the scope of the present invention include, by way of non-limiting example: age-related cataracts, senile cataract, Alzheimer's Disease (AD), Familial AD, Sporadic AD, Creutzfeld-Jakob disease, variant Creutzfeld-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Down's Syndrome (Trisomy 21), Pick's Disease (Frontotemporal Dementia), Lewy Body Disease, neurodegenration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, Pick's disease, corticobasal degeneration, hereditary frontotemporal dementia (with or without Parkinsonism), and Guam amyotrophic lateral sclerosis/parkinsonism dementia complex). These disorders may occur alone or in various combinations. For example, individuals with AD are characterized by extensive accumulation of amyloid in the brain in the form of senile plaques, which contain a core of amyloid fibrils surrounded by dystrophic neurites. Some of these patients exhibit clinical signs and symptoms, as well as neuropathological hallmarks, of Lewy Body disease.

As used herein, “Aβ-metal complex” proteins encompass metal atoms bound or coordinated by β-amyloid precursor protein (APP), Aβ, or a fragment thereof (e.g., Aβ₁₋₄₀, Aβ₁₋₄₂), prion proteins, and synuclein. In addition, “Aβ-metal complexes” or conjugates may also comprise one or more addition proteins or polypeptide components including, for example, α-, β-, and/or γ-crystallin.

In certain aspects, the present invention relates to methods for the detection and measurement of Aβ-metal complexes in the ocular lens of a subject for the detection and monitoring of amyloid-related disease progression through the use of non-invasive Raman spectroscopic techniques. An embodiment of this aspect, encompasses a method of detecting, diagnosing, prognosing, staging, and/or monitoring a mammalian amyloidogenic disorder or a predisposition thereto carried out by detecting a protein or polypeptide aggregate in at least one of the cortical, nuclear, peripheral cortical fibers, or supranuclear region of an ocular lens of a subject, for example, a mammal such as a human. This determination is compared to or normalized against measurements taken from the same region of the same lens where more general effects of aging are observed. In certain embodiments, the methods include comparing an instant Aβ-metal determination to measurements taken from the same region of the same lens of the same subject acquired at one or more previous time points.

In other embodiments, comparisons are made to a population norm, e.g., data compiled from a pool of subjects with and without amyloid-related disease. The presence of or an increase in the amount of Aβ-metal complexes in the supranuclear and/or cortical lens regions of the test subject compared to a normal control value indicates that the test subject is suffering from, or is at risk of, developing an amyloid-related disorder. A normal control value corresponds to a value derived from testing an age-matched subject who is known to not exhibit or present amyloid-related disease or a value derived from a pool of normal, healthy (non-AD) individuals.

The Raman spectroscopic technique is used to non-invasively detect and quantitate lens Aβ-metal complexes or conjugates. A significant advantage of the methods described herein is the ability to, at an earlier stage than previously possible, specifically, reliably, and non-invasively diagnose cataracts, and AD antemortem. Prior to the instant invention, no reliable antemortem diagnostic methods were available that could detect the presence of amyloid proteins before they agglomerate and/or accumulate into fibrils large enough to scatter light (e.g., U.S. Pat. No. 7,107,092 to Goldstein). Based on the discovery that an increase in Aβ-metal complexes or conjugates is detectable the ocular lens of a cataract and AD patient compared to normal lenses, early detection of both cataracts and neurodegeneration is possible. Thus, another advantage of the method is detection of a pathologic state (or pre-pathologic state) prior to any clinical indication of disease, e.g., impaired vision and/or cognition. In addition, the cataract phenotype observed in the supranuclear/peripheral cortical region is closely associated with neuropathologically confirmed AD. This supranuclear/cortical cataract is distinct from the much more common age-related cataract, which is found in the lens nucleus. A further advantage to this technique is the ability to monitor disease progression as well as responsiveness to therapeutic intervention. Another advantage of the instant methods is that the amount and rate of progression of Aβ-metal accumulation and aggregation in the eye closely parallels disease progression in the brain, providing an accurate and reliable determination of pathology in an otherwise inaccessible tissue.

The presence and/or an increase in the amount of Aβ-metal complex detected in a subject's eye tissue over time indicates a poor prognosis for disease, whereas absence or a decrease over time indicates a more favorable prognosis. For example, a decrease or decrease in the rate of accumulation in Aβ-metal complex or similar changes in the associated ocular morphological features in eye tissue after therapeutic intervention indicates that the therapy has clinical benefit. Examples of therapeutic approaches that have demonstrated efficacy for amyloid-related disorders includes, for example, drug therapy such as administration of a secretase inhibitor, vaccine, antioxidant, anti-inflammatory, metal chelator, or hormone replacement or non-drug therapies.

In any of the embodiments described herein, the subject to be tested can include, for example, a mammal, a human, as well as other animals such as dogs and cats, livestock such as cows, sheep, pigs horses, and the like.

In another embodiment, the invention includes methods for determining the predisposition of a subject to an amyloid-related disorder. The method comprising the steps of administering a diagnostic test to measure and detect Aβ-metal complex in the ocular lens of a subject that has a positive family history of cataracts, familial AD or other risks factors for AD (such as advanced age), or is suspected of suffering from an amyloid-related disorder, e.g., by exhibiting impaired cognitive function, or is at risk of developing such a disorder; and comparing the test measurements with those taken previously from the same subject, from one or more normal subjects, either within the same family or from a database of normal subjects of similar age. Subjects at risk of developing such a disorder include elderly patients, those who exhibit dementia or other disorders of thought or intellect, or patients with a genetic risk factor. Many genetic predisposition or high risk loci are well known in the art and would be known by those of skill in the art.

An amyloid-related disease state is indicated by the presence of Aβ-metal complexes or conjugates in the ocular lens of the subject in at least one region of the lens, for example, in the cortical region peripheral cortical region, nuclear region or supranuclear or cortical region of the lens. For example, the amount of Aβ-metal complex is increased in a disease state compared to a normal control amount, i.e., an amount associated with a non-diseased individual.

According to the methods of the invention, Aβ-metal complexes or conjugates are detected non-invasively, i.e., using a device or apparatus that is not required to physically contact ocular tissue. For example, the invention includes a method of diagnosing an amyloid-related disorder or a predisposition thereto in a mammal, by illuminating the lens tissue of the subject tissue with an excitation light beam and detecting scattered, reflected or other light signal emitted from the tissue. In the methods described herein, Aβ-metal complexes or conjugates are detected using Raman spectroscopic techniques (described in detail below).

In another embodiment, the invention includes methods of monitoring the efficacy of a therapeutic agent or intervention for an amyloid-related disease or disorder by course, for example, before therapy begins and at various times after (or during) therapeutic intervention. An increase in the amount or rate of accumulation of Aβ-metal complexes or conjugates indicates a less favorable prognosis or less favorable response to therapy, whereas a decrease in the amount or rate indicates a favorable response to therapy or a favorable prognosis. For example, a pre-treatment status of the patient is determined, the patient is treated, and then the patient's condition is followed using Raman techniques. An increase in the amount or rate of formation of Aβ-metal complexes or conjugates or accumulation of amyloid-related protein or peptides is compared to a normal control value or a prior measurement in the same subject.

Detection of protein aggregation or accumulation or deposition of amyloid-related proteins and/or Aβ-metal complexes or conjugates in the ocular lens is ratiometrically, volumetrically, or otherwise mathematically compared to the same or similar measurements in the nuclear or other regions of the lens. The methods are useful to measure protein aggregation or accumulation or deposition of Aβ-metal complexes or conjugates in other ocular tissues, including but not limited to the cornea, the aqueous humor, the vitreous humor, and the retina. Raman spectroscopy provides a new method of cataract diagnosis based on identification at the molecular level and has significant potential to detect critical changes, which precede significant scattering or amyloid formation.

Raman Laser Spectroscopy Methods.

Raman spectroscopy is a spectroscopic technique used in condensed matter physics and chemistry to study vibrational, rotational, and other low-frequency modes in a system. It measures the frequency spectrum of light scattered from a material. A monochromatic light (laser source) of a given frequency is used to illuminate and excite molecules within a material that have specific internal vibration frequencies due their molecular structure. The result of this excitation is that two types of scattered light are produced. One is called Rayleigh scattering, which is strong and has the same frequency as the incident beam (elastic scattering). The other is called Raman scattering which although is much weaker in intensity, directly reflects the vibrational, rotational or electronic energy specific for each molecule. Therefore, Raman spectra provide specific chemical signatures for each molecule in the sample. The Raman spectrum of each different molecule has its unique quality and characteristics and allows for identification of two different molecules in a mixture. To obtain spectra of Raman scattering, wavelengths close to the laser line (elastic scattering) are filtered, and those in a spectral window away from the laser line are recorded by a detector.

The Raman effect occurs when light impinges upon a molecule and interacts with the electron cloud of the bonds of that molecule. A molecular polarizability change, or amount of deformation of the electron cloud, with respect to the vibrational coordinate is required for the molecule to exhibit the Raman effect. The amount of the polarizability change will determine the intensity, whereas the Raman shift is equal to the vibrational level that is involved. The incident photon (light quantum), excites one of the electrons into a virtual state.

Raman spectroscopy works as follows: light hits a substance, causing the atoms in the substance to vibrate sympathetically. The collision of photons with the substance causes some of the photons to gain or lose energy, resulting in a secondary light of a different wavelength. It relies on inelastic scattering, or Raman scattering of monochromatic light, usually from a laser in the visible, near infrared, or near ultraviolet range. The laser light interacts with phonons or other excitations in the system, resulting in the energy of the laser photons being shifted up or down. The shift in energy gives information about the phonon modes in the system. Infrared spectroscopy yields similar, but complementary information. Typically, a sample is illuminated with a laser beam. Light from the illuminated spot is collected with a lens and sent through a monochromator. Wavelengths close to the laser line (due to elastic Rayleigh scattering) are filtered out and those in a certain spectral window away from the laser line are dispersed onto a detector.

Spontaneous Raman scattering is typically very weak, and as a result the main difficulty of Raman spectroscopy is separating the weak inelastically scattered light from the intense Rayleigh scattered laser light. Raman spectrometers typically use holographic diffraction gratings and multiple dispersion stages to achieve a high degree of laser rejection. In the past, PMTs were the detectors of choice for dispersive Raman setups, which resulted in long acquisition times. However, the recent uses of CCD detectors have made dispersive Raman spectral acquisition much more rapid. Raman spectroscopy has a stimulated version, analogous to stimulated emission, called stimulated Raman scattering. For the spontaneous Raman effect, the molecule will be excited from the ground state to a virtual energy state, and relax into a vibrational excited state, and which generates Stokes Raman scattering. If the molecule was already in an elevated vibrational energy state, the Raman scattering is then called anti-Stokes Raman scattering.

Raman spectroscopy is commonly used in chemistry, since vibrational information is very specific for the chemical bonds in molecules. It therefore provides a fingerprint by which the molecule can be identified. The fingerprint region of organic molecules is in the range 500-2000 cm⁻¹. Another way that the technique is used is to study changes in chemical bonding, e.g. when a substrate is added to an enzyme.

Laser Raman spectroscopy is a powerful structural biochemistry technique which can safely provide information about regional lens hydration status (3417 cm⁻¹), the lens water:protein ratio (expressed as Raman intensity ratio at 3417 cm⁻¹:2936 cm⁻¹), oxidation state of lens thiols, and the hydrogen bond microenvironment of the aromatic amino acid residues tryptophan (bands at 881 and 760 cm⁻¹) and tyrosine (doublet near 840 cm-1). All of these factors are altered during cataractogenesis. Changes in the hydrogen-bonding microenvironment of tyrosine residues are particularly intriguing since dityrosine formation may be an important factor in Aβ aggregation. The instrument operating in the Raman spectroscopy mode is also used to detect and quantitate specific Aβ-lens protein associated Raman signature signals. For example, since Aβ has a high affinity for redox-active metal ions. Such interactions with these metals are involved in Aβ-lens protein aggregation. The Raman spectra is used in the 1750-720 cm⁻¹ interval to detect Aβ-metal coordination.

The fiber-optic probe is also used in a Raman scattering, or related Raman methodology mode. In laser Raman spectroscopy, a monochromatic laser light is directed onto a particular target material to be tested. For example, the bean is directed to the supranuclear region of the lens. A detection system then detects light returning, or scattered, from the target. The majority of the light returning from the material is scattered elastically at the same wavelength of the original projected laser light (Rayleigh scattering). A subset of the light returning from the material is scattered inelastically at a wavelength different from that of the original projected laser light in a manner known as Raman scattering. Raman scattered light is then separated from Rayleigh scattered light with the use of filters, optical gratings, prisms, and other wavelength selection techniques.

The energy difference between Raman and Rayleigh scattered light is related to the vibrational, rotational, or liberational states, or mixtures thereof, of various molecules in the material being evaluated. Each of the peaks in the resulting Raman spectrum corresponds to a particular Raman active vibration of a molecule or a component thereof. The Raman energy shift is independent of the wavelength of the directed laser light. The energy difference corresponding to the elastically and inelastically scattered light for a particular material remains constant for that material.

Raman spectroscopy techniques offers several advantages in the instant methods over traditional light scattering techniques. For example, water produces very weak RS scattering, and therefore, RS spectra from biological samples can be obtained without major interference from water. In addition, since the diameter of the taster light is very small RS spectra can be obtained from small, specifically targetted regions. Furthermore, RS spectra show very specific bands which help to identify and quantify a molecule easily. Next, since each biological molecule has its own unique RS spectrum, RS allows several molecules to be individually identified in a mixture.

Currently, Raman spectroscopy is used for several applications in the eye which will aid in its development for detection and analysis of Aβ-metal complexes in the eye. These applications include analysis of antibiotic concentration and for glucose in diabetics in the anterior chamber, and has been developed for analysis of pigments at the back of the eye in the retina.

The data from Raman scattering is used to locate, identify and quantitate concentrations of a material. For example, the Raman fingerprint of an Aβ aggregate is different from that of an aggregrate associated with an age-related nuclear cataract by virtue of i) signal localization within the lens (supranuclear/cortical versus nuclear), and ii) Aβ-lens protein and Aβ-metal interaction (and interactions between Aβ, other lens proteins, and metals).

The absolute intensities of the resulting Raman peaks are directly related to the concentration of the Raman-active molecules in the material. The fingerprints are characterized by distinct spectral positions, signal strengths, and spectral widths. For example, a low power laser light in the range of 450-550 nm or is directed to the target region of the eye. Scattered light is optionally routed to a spectrally selective system, which selects only the Raman scattered light and rejects the Rayleigh scattered light to allow analysis of Raman signals absent interference from Rayleigh signals. Methods and devices for spectrally selecting scattered light are known in the art, e.g., grating monochromators, holographic filters, prisms, dielectrics, or combinations thereof.

A filter may be placed on both monomode optical fibers to allow only one frequency of light to be emitted or detected. The detected light is converted using a digital correlator into a spectrum that serves as a signature to detect protein aggregation. Interatomic vibration frequencies are recognized and assigned to specific protein aggregations. Using the techniques of Raman scattering, or related Raman methodology, the protein composition of an ocular aggregate is identified. An emission signature or Raman spectra, which indicates the presence of an Aβ aggregate, an Aβ-α crystallin aggregate, Aβ-β crystallin aggregate, or a Aβ-γ crystallin aggregate indicates a diagnosis of cataracts, Alzheimer's Disease, or a predisposition to developing the disease or an amyloid disorder.

In solid state physics, spontaneous Raman spectroscopy is used to, among other things, characterize materials, measure temperature, and find the crystallographic orientation of a sample. As with single molecules, a given solid material has characteristic phonon modes that can help an experimenter identify it. In addition, Raman spectroscopy can be used to observe other low frequency excitations of the solid, such as plasmons, magnons, and superconducting gap excitations.

Raman spectroscopy offers several advantages for microscopic analysis. Since it is a scattering technique, specimens do not need to be fixed or sectioned. Raman spectra can be collected from a very small volume (<1 μm in diameter); these spectra allow the identification of species present in that volume. Water does not interfere very strongly. Thus, Raman spectroscopy is suitable for the microscopic examination of minerals, materials such as polymers and ceramics, cells and proteins. A Raman microscope begins with a standard optical microscope, and adds an excitation laser, a monochromator, and a sensitive detector (such as a charge-coupled device (CCD) or photomultiplier tube (PMT)). FT-Raman has also been used with microscopes.

In direct imaging, the whole field of view is examined for scattering over a small range of wavenumbers (Raman shifts). For instance, a wavenumber characteristic for cholesterol could be used to record the distribution of cholesterol within a cell culture.

The other approach is hyperspectral imaging or chemical imaging, in which thousands of Raman spectra are acquired from all over the field of view. The data can then be used to generate images showing the location and amount of different components. Taking the cell culture example, a hyperspectral image could show the distribution of cholesterol, as well as proteins, nucleic acids, and fatty acids. Sophisticated signal- and image-processing techniques can be used to ignore the presence of water, culture media, buffers, and other interferents.

Raman microscopy, and in particular confocal microscopy, has very high spatial resolution. For example, the lateral and depth resolutions were 250 nm and 1.7 μm, respectively, using a confocal Raman microspectrometer with the 632.8 nm line from a He—Ne laser with a pinhole of 100 μm diameter. Since the objective lenses of microscopes focus the laser beam to several micrometres in diameter, the resulting photon flux is much higher than achieved in conventional Raman setups. This has the added benefit of enhanced fluorescence quenching. However, the high photon flux can also cause sample degradation, and for this reason some setups require a thermally conducting substrate (which acts as a heat sink) in order to mitigate this process.

By using Raman microspectroscopy, in vivo time- and space-resolved Raman spectra of microscopic regions of samples can be measured. As a result, the fluorescence of water, media, and buffers can be removed. Consequently in vivo time- and space-resolved Raman spectroscopy is suitable to measure cells, proteins, organs, and erythrocytes. Raman microscopy for biological and medical specimens generally uses near-infrared (NIR) lasers (785 nm diodes and 1064 nm Nd:YAG are especially common). This reduces, the risk of damaging the specimen by applying high power. However, the intensity of NIR Raman is low (owing to the ω⁻⁴ dependence of Raman scattering intensity), and most detectors required very long collection times. Recently, more sensitive detectors have become available, making the technique better suited to general use. Raman microscopy of inorganic specimens, such as rocks and ceramics and polymers, can use a broader range of excitation wavelengths.

Several variations of Raman spectroscopy have been developed and that can be used in the methods of the invention. The usual purpose is to enhance the sensitivity (e.g., surface-enhanced Raman), to improve the spatial resolution (Raman microscopy), or to acquire very specific information (resonance Raman).

Surface Enhanced Raman Spectroscopy (SERS)—Normally done in a silver or gold colloid or a substrate containing silver or gold. Surface plasmons of silver and gold are easily excited by the laser, and the resulting electric fields cause other nearby molecules to become Raman active. The result is amplification of the Raman signal (by up to 10¹¹). This effect was originally observed by Fleishman but the prevailing explanation was proposed by Van Duyne in 1977.

Hyper Raman—A non-linear effect in which the vibrational modes interact with the second harmonic of the excitation beam. This requires very high power, but allows the observation of vibrational modes which are normally “silent”. It frequently relies on SERS-type enhancement to boost the sensitivity.

Resonance Raman spectroscopy—The excitation wavelength is matched to an electronic transition of the molecule or crystal, so that vibrational modes associated with the excited electronic state are greatly enhanced. This is useful for studying large molecules such as polypeptides, which might show hundreds of bands in “conventional” Raman spectra. It is also useful for associating normal modes with their observed frequency shifts.

Spontaneous Raman Spectroscopy—Used to study the temperature dependence of the Raman spectra of molecules.

Optical Tweezers Raman Spectroscopy (OTRS)—Used to study individual particles, and even biochemical processes in single cells trapped by optical tweezers.

Stimulated Raman Spectroscopy—A two color pulse transfers the population from ground to a rovibrationally excited state, if the difference in energy corresponds to an allowed Raman transition. Two photon UV ionization, applied after the population transfer but before relaxation, allows the intra-molecular or inter-molecular Raman spectrum of a gas or molecular cluster (indeed, a given conformation of molecular cluster) to be collected. This is a useful molecular dynamics technique.

Spatially Offset Raman Spectroscopy (SORS)—The Raman scatter is collected from regions laterally offset away from the excitation laser spot, leading to significantly lower contributions from the surface layer than with traditional Raman spectroscopy.

(For review of Raman spectroscopic techniques, see: Ellis, D. I. and Goodacre, R., Metabolic fingerprinting in disease diagnosis: biomedical applications of infrared and Raman spectroscopy. Analyst, 131, 875-885 (2006); and P. Matousek, et al., Subsurface Probing in Diffusely Scattering Media using Spatially Offset Raman Spectroscopy. Appl. Spectrosc. 59, 393 (2005); the teachings of which are hereby incorporated by reference in their entirety).

The following, non-limiting, examples illustrate methods of detecting ocular Aβ-metal complexes and the use of such methods to diagnose, monitor and stage cataracts and neurodegenerative disorders.

Additional objects and advantages of the present invention will be appreciated by one of ordinary skill in the art in light of the current description and examples with reference to the drawings, and are expressly included within the scope of the present invention.

### Examples and Methods

A fundamental goal for diagnosing and preventing major age-dependent disease of lens, senile cataract, is to identify key cell biology processes involved, and then work to understand how these processes are subverted by stress and aging in ways that specifically contribute to disease phenotypes. We have identified key biomarkers and technology to diagnose early lens and AD pathology that also provide critical insight into cataract mechanisms. Our previous work demonstrated that extensive ‘neuron-specific’ cell biology processes in neurons centered on the normal role of the β-amyloid precursor protein (APP) are shared with the lens to a striking degree, beginning early in embryogenesis and continuing in adulthood. These studies led us to demonstrate that basic mechanisms of Alzheimer disease (AD) pathology are fundamentally involved in cataract.

Moreover, biomarker increases in Aβ are a key element in the hallmark accumulation of Copper (Cu) in cataracts. Aβ pathology has now been demonstrated in human cataract and in each of several cataract animal models examined, and conversely, specific cataract formation has been demonstrated in human AD donor lenses and in transgenic AD mouse models. This has led to an important understanding that AD animal models provide fundamentally germane models of age-dependent cataract formation, and build on generalized stress models of senile cataract that have been predominant in cataract research. A central event in AD is the production of Aβ peptides and their interaction with metals that produces oxidative stress and determines Aβ aggregation and the formation of deposits. Together, these studies lead to our overall hypothesis that lens and brain extensively share the same biological diathesis model for age-dependent disease, and that Aβ production in lens is a primary and consistent diagnostic biomarker that can be used for the diagnosis of senile cataract, and further that lens Aβ pathology also provides a critical biomarker of Aβ pathology in brain for diagnosis of AD in human patients. The specific monitoring of Aβ-metal complexes in the lens provides an important new tool for diagnosing early changes in cataract formation and monitoring Aβ in the lens for cataract detection, and provides an easily accessible and sensitive method for monitoring early development of AD pathology in brain.

Our work provides strong evidence that strictly non-invasive Raman laser spectroscopy can provide an important new tool for detecting early stages of cataract formation and AD pathology. Raman spectra demonstrate characteristic hallmark changes in Aβ-metal complexes that are fundamentally germane biomarkers of AD pathology in lens that also provide critical diagnostic information about corresponding production of AD pathology in brain. Because Raman spectroscopy identifies Aβ-metal complexes at the molecular level in situ, this method can detect early small soluble Aβ forms in the transparent lens that appear before significant light scattering is detected and before detection of significant higher order amyloid 2° protein structure.

Aβ Fibril and amyloid protein formation. Several lines of evidence indicate that a key seminal event in the formation of AD pathology is the production of Aβ peptides that can self associate to form hallmark amyloid stacked β-sheet protein fibrils. These aggregates occur in senile plaques in human brain, and in cataractous regions in age-related cataract in the lens. However, Aβ production and aggregate formation in brain or lens can occur in locations that may not affect function. Evidence indicates that Aβ aggregation is determined in large part by metal interactions, particularly with Cu and Zn. However Cu and Zn can produce different complexes with Aβ. Iron occurs in Aβ deposits but appears not to form similar complexes. Zn only contributes to Aβ aggregation and fibril formation, whereas Cu promotes fibril formation, but also can form small soluble redox active Aβ-Cu complexes that oxidize cellular component substrates and produce hydrogen peroxide (H₂O₂).

Consistent with Zn not being a transition metal, Aβ-Zn complexes are redox inert. Under certain pH conditions Aβ-Cu complexes also produce fibrils and aggregates that are also largely redox incompetent. Aβ oxidation of lens proteins has-been demonstrated in vitro and evidence indicates this is also metal and/or Cu dependent. Aβ interaction with metals and its affects on aggregation have been determined in biophysical sedimentation and turbidity assays. A variety of molecular spectroscopy methods have also been used to specifically characterize Aβ-metal interactions and confirm data from biophysical experiments.

The present invention relates to the novel application of Raman spectroscopy for detecting and characterizing Aβ-metal complexes at the molecular level in the intact lens directly related to cataract and Alzheimer pathology in brain. Aβ peptides have a very high affinity for Cu, and Cu readily displaces Zn in Aβ complexes. Aβ aggregates are hallmarks of AD pathology in brain and have been demonstrated in the heavily studied AD transgenic (Tg) mouse model, Tg2576. However, fibrils and plaques are characteristic of later disease stages. Thus, identification of small soluble Aβ-metal complexes with Raman spectroscopy has significant potential to provide insights into disease mechanism and diagnostic information about early disease stages in lens and brain.

Small soluble Aβ-Cu complexes are produced early-on in the amyloid-related disease process and exist as monomers or small soluble oligomers. Evidence indicates that monomers or perhaps also dimers acquire redox competent SODI-like enzymatic activity that produces H₂O₂ to create oxidative stress, which disrupts cell function and contributes to cell death. Thus, small non-aggregated Aβ forms appear early in the disease and appear to be more toxic. Consistent with this, removing metals with chelators impairs Aβ toxicity, and metal chelators are currently in human trials as potential AD treatments, and for cataract. Conversely, Aβ metal interactions and fibril formation have also been termed Aβ ‘entombment.’ However Aβ in fibrils may still represent a toxic store of Aβ peptides and there is evidence that Aβ aggregates occur in dense regions or plaques in brain and in lens opacities in human and animal lens cataracts. A third key mechanism ascribed to Aβ metal binding is the disruption of metal homeostasis and directly implicates Aβ in Alzheimer cellular and systemic pathophysiology.

APP holoprotein is also fundamentally involved in systemic Cu physiology in the body. In addition to Aβ, a second Cu-binding site in the N-terminus of APP is required for normal Cu efflux from cells, and has a critical role in Cu metabolism throughout the body. Key data consistent with the important role of systemic factors that include diet and hormonal effects in AD comes from APP knockout mice. These mice have 40% increased Cu in brain, but an even greater (80%) increase in liver. However, the role of Aβ in cellular and systemic Cu homeostasis is not known but recent studies document a >25% increase in serum Cu in AD patients. The present examples demonstrate the relationship between Aβ-metal interactions governing Aβ monomeric, oligomeric and fibril formation, and the structure of Aβ-Cu redox active complexes which can oxidize cellular components.

Histidine Aβ residues have key roles in metal chelation. Of the 20 common amino acids, Histidine (H or His) has the highest affinity for Cu and other metals. For example, nickel columns are used routinely to purify His-tagged proteins. However, the molecular environment of His residues, as well as coordination with other chemical groups, significantly alters peptide metal affinities.

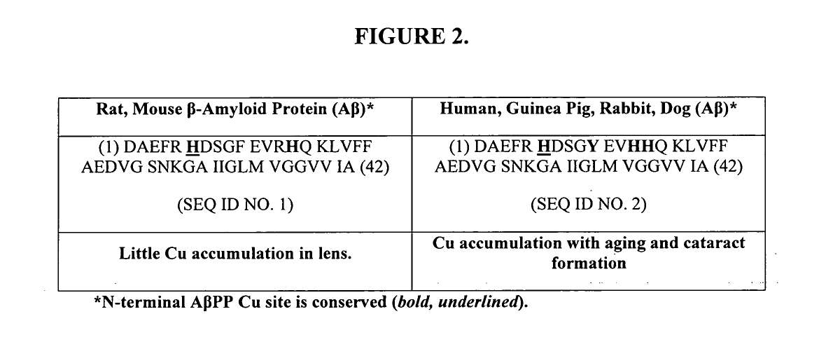

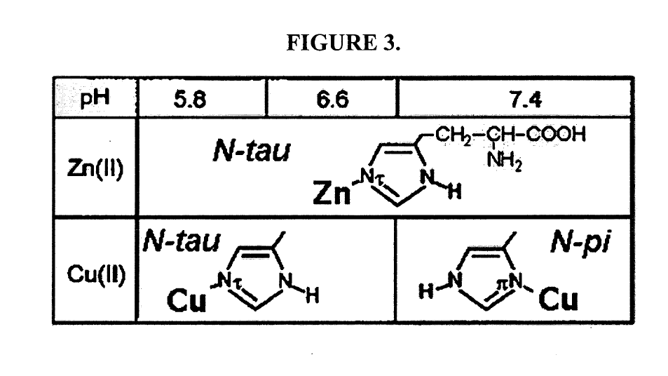

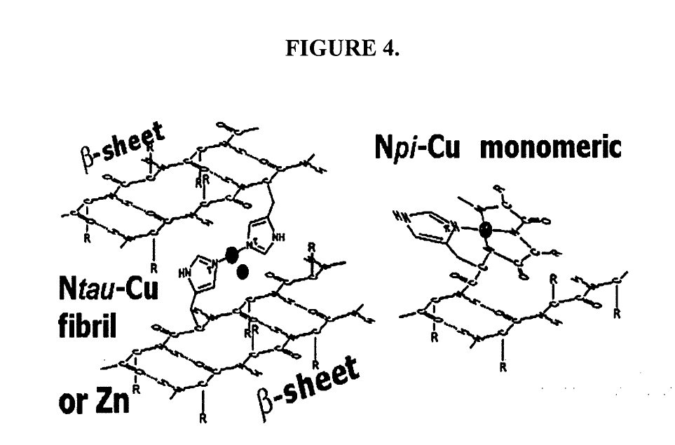

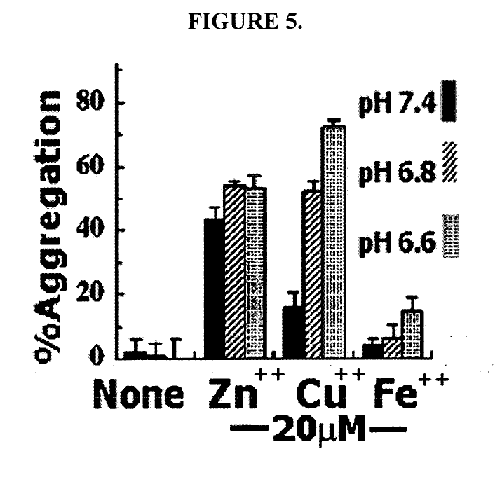

Histidine at position 14 of Aβ (FIG. 2; SEQ ID NOs.: 1 and 2) in most mammals, including rat and mouse, is thought to provide critical initial interactions with Cu and Zn. These metals can bind at one of two nitrogens in the His imidazole ring (FIG. 3). The nitrogen proximal to the peptide bond is referred to as N-pi and the distal nitrogen as N-tau. In vitro, studies indicate Cu and Zn-mediated Aβ aggregation and fibril formation occurs with Cu or Zn binding to N-tau (FIG. 4) which is conducive with interactions with N-tau groups on adjacent peptides, for inter-Aβ peptide associations and fibrils. In contrast N-pi binding favors intra-Aβ peptide binding and monomeric Aβ-Cu, also modeled in FIG. 4. Both models are supported by Raman and X-ray fluorescence spectroscopy data. N-tau Cu or Zn binding is thought to allow the remaining portion of the Aβ peptide to form characteristic protein parallel β-sheets that can ‘stack’ to form hallmark amyloid protein structure. Biophysical analysis for Aβ-metal aggregate formation agrees with Raman spectroscopy data indicating Zn only produces aggregation regardless of the pH (FIG. 5), whereas Cu forms small soluble Aβ at physiological pH, but leads to aggregation and fibril formation at slightly acidic pH.

Raman spectroscopy and biophysical data indicate Cu readily displaces Zn in Aβ complexes, however with a pH dependence. At physiological pH, Cu and Zn bind equally. At pH 6.8 Cu completely displaces Zn from Aβ. Both Cu increases and mild acidosis has particular significance in lens. Although rat and mouse Aβ do not have two key metal chelating amino acids and have much lower metal affinities, the capacity of these peptides to produce H₂O₂ is not as severely crippled as might be expected. In vitro, rat Aβ₁₋₄₂ produced similar amounts of H₂O₂ as human Aβ₁₋₄₀, and rat Aβ₁₋₄₀ produced ˜20% of that level, consistent with mAPP mice that also exhibit significant decreases in viability. Further, a second important point is that although Cu does not increase in rat lenses, atomic absorption spectrophotometry studies measure decreased Zn with constant Cu in aging rat lenses and cataract, thus a similar effect is produced with a relative increase in Cu.

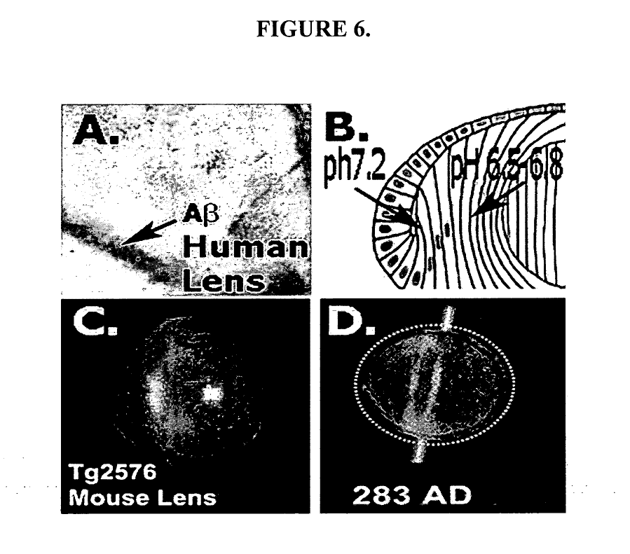

The lens periphery normally undergoes mild acidosis towards the lens interior, entirely consistent with lens APP expression patterns and the formation of lens pathology involving Aβ, examined to date in supranuclear cataracts in Tg2576 AD transgenic mouse lenses and in lenses from human AD donors. Our previous studies demonstrated Aβ expression is relegated to the peripheral cortical fibers in human lenses as well as in rat and monkey lenses, and in monkey lenses exposed to oxidative stress where APP and Aβ levels increase. This lens region (FIG. 6B) is where a significant decline in pH normally occurs in lenses. Near the lens surface at the equatorial margin the pH is ˜7.2-7.4. As one moves into the lens, the pH significantly decreases to approximately pH 6.6-6.8. The rapid pH decrease completes near the border of the lens outer cortex and the inner lens region termed the lens nucleus which has been termed the “supranuclear” region. It is at this supranuclear border that increased lens opacification has been described in Tg2576 transgenic mouse lenses and in cataracts which have been associated with AD in humans described above. Although variable, particulate light scattering (opacities) visible in Tg2576 mouse lens in FIG. 6C, and a human lens (FIG. 6D) is consistent with the hallmark accumulation of Cu (and not Zn) that occurs in cataract and is consistent with the pH dependence of Aβ-Cu complex solubility profile shown in biophysical and spectroscopy analysis of pH dependence of Aβ aggregation and the role of Aβ-metal binding that determines Aβ aggregation. The data agree that Aβ-metal complexes in lenses are more likely to be in small soluble forms near the periphery, and contribute to Aβ aggregation a few hundred micrometers into the lens near the supranuclear border. We note that, likely due to high Aβ metal affinity, Aβ is predominantly isolated with bound Cu or other metals. Although Cu is most often not free in cells, significant Cu movement occurs in cells and has been reviewed elsewhere.

In vitro characterization of Aβ Cu and Zn interactions. Raman spectroscopy has proved to be a particularly useful tool to investigate the Cu and Zn binding site on the histidyl imidazole ring. Raman spectra also provide a strong signal characteristic of amide bond interactions in protein n-sheet secondary structure. Aβ peptides in solution have been mixed with Cu and Zn and Raman spectra have been taken. In this way specific amide signature intensities in the Raman spectra have been identified that provide direct information about Aβ peptide stacked β-sheets that form fibrils (AMIDE I). Companion data from other spectroscopy methods including X-ray absorption spectroscopy and synchrotron Fourier transform infrared micro-spectroscopy (FTIRM) has provided further support for these findings regarding Aβ metal interactions, and have been used to characterize amyloid peptides in plaques in brain tissue of AD patients provided evidence of Aβ metal complexes derived from Raman studies.

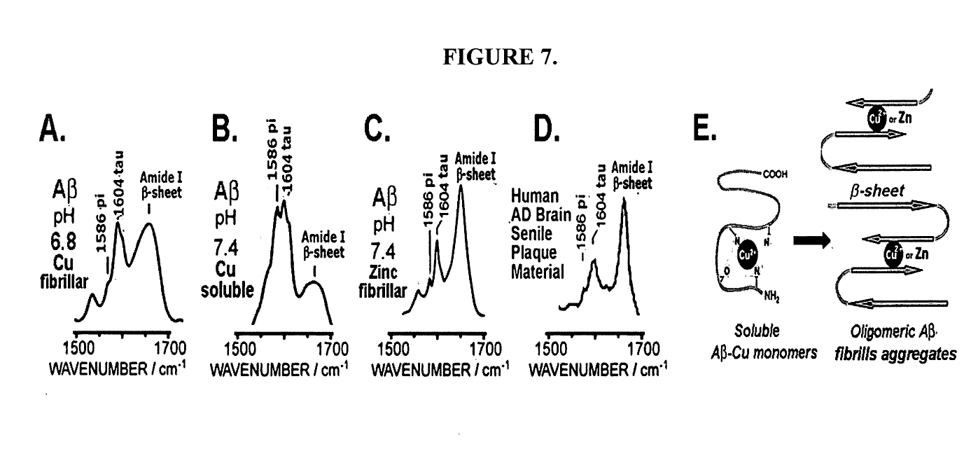

Raman spectroscopy identifies signatures of N-tau, N-pi and Amide I Aβ metal interactions. FIGS. 7A and 7B show the region from 1500-700 cm⁻¹ for Aβ incubated with Cu. In panel A, the mildly acidic pH conditions similar to that found at the ‘supranuclear’ border of lenses promotes Cu to displace whatever Zn may be present. In addition peak intensity at 1604 cm⁻¹ is observed, with a much smaller “shoulder” at ˜1586 cm⁻¹. Peak intensity at 1604 cm⁻¹ is a signature of N-tau interactions with Histidine imidazole nitrogens, and leads to inter-Aβ peptide interactions, fibril formation, and aggregation. Also consistent with this, the Amide I band is also increased indicative of stacked β-sheet and fibril formation. In contrast, Cu Aβ interaction at pH 7.4 shows relatively equal peak intensities at 1604 cm-1 and 1586 cm-1 indicating strong Cu interaction at the N-pi Nitrogen as well. Correspondingly, the Amide I peak is decreased. In FIG. 7C, Zn interaction with Aβ is similar to that observed with Cu at physiological pH. Again the Amide I band is consistent with Zn-promoted fibril formation and significant stacked β-sheet.

FIG. 7D shows data from Dong et al. (2003). Those investigators obtained material from senile plaques from brain tissue of AD affected individuals. Significantly the Raman spectra profile in this region is remarkably similar to results obtained with purified Aβ in vitro. Significant stacked β-sheet structure is indicated and Aβ-Cu or —Zn is identified by the peak intensity at 1604 cm-1. We note that peak intensities vary ±5 cm.⁻¹

Raman spectroscopy has been used to analyze lens structure for over 35 years. Numerous Raman spectroscopy studies since the '70s repeatedly demonstrated that the lens interior has a protein structure that is overwhelmingly comprised of stacked β-sheet protein, with polarized Raman spectroscopy determining that lens pleated β-sheets are organized in ordered parallel arrays. This suggests that the lens interior fits many of the essential requirements to qualify as a predominantly amyloid-like protein structure as indicated in our previous study. In that study, in vitro amyloid dye binding studies, performed using FPLC purified crystallins, indicated that the major β-crystallins in particular, which are highly concentrated in the lens interior, have a significant capacity to form amyloid β-sheet structure identifiable with well-characterized thioflavine fluorescence in vitro, using methods described by LeVine for analysis of Aβ peptides. We note Amide I signature and those for Aβ-metal complexes are distinct in Raman spectra in FIG. 7 and in our preliminary analysis of lens below. The present Raman spectroscopy method for detecting Aβ-complexes is not affected by native lens protein secondary structure that appears to be fundamentally related to Aβ peptide fibrils and secondary protein structure detected in the same way, and currently developed for AD diagnosis by others.

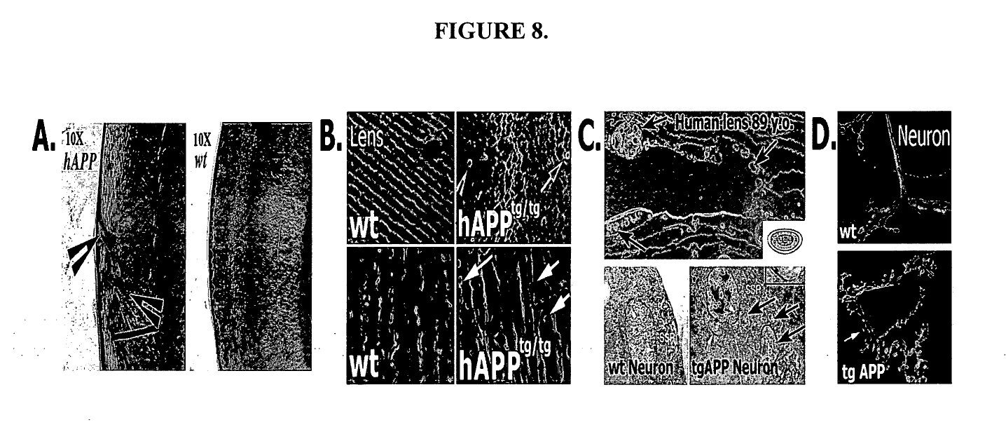

AβPP and AD biology has a fundamental role in lens development and cargo vesicle trafficking cell biology beginning at early stages of embryonic development and continuing in adult lenses. We discovered the remarkable degree to which Alzheimer protein biology is central to both lens fiber cells and neurons. One of the earliest sites of embryonic APP expression is the posterior lens vesicle in E11 rat (and chick). This site is where dramatic fiber cell elongation initiates to produce mature fiber cells that approach ˜1 cm in length in humans and rabbits (FIG. 8).

The normal cell biology and associated factors for APP linking of synaptic and cargo vesicles to cytoskeleton for trafficking are remarkably conserved in lens, and increased APP expression in lens fiber cells produces intracellular vesicles in APP Tg lenses similar to neuronal phenotypes. An editorial accompanying our study of a Yeast Artificial Chromosome (YAC) hAβPP Tg mouse lenses stated “the data clearly demonstrate that AβPP is a key element in the development of fiber cell formation and early-onset cataracts in DS. Moreover, the results suggest that AβPP and other factors involved in AD contribute to the pathogenesis of age-related cataracts in the general population.” However, we understand that cataractogenesis, similar to early-AD in DS, clearly involves many factors. Our group began to study the lens in AD animals models by examining YAC single copy human APP Tg mouse model, as a first step in examining AD biology, and to begin to investigate causal associations with pathology in lens. Our view is this model better obviates interpretations of gratuitous effects that may occur in APP Tg2576 and other mouse models over-expressing APP in brain or lens expressed by heterologous promoters.

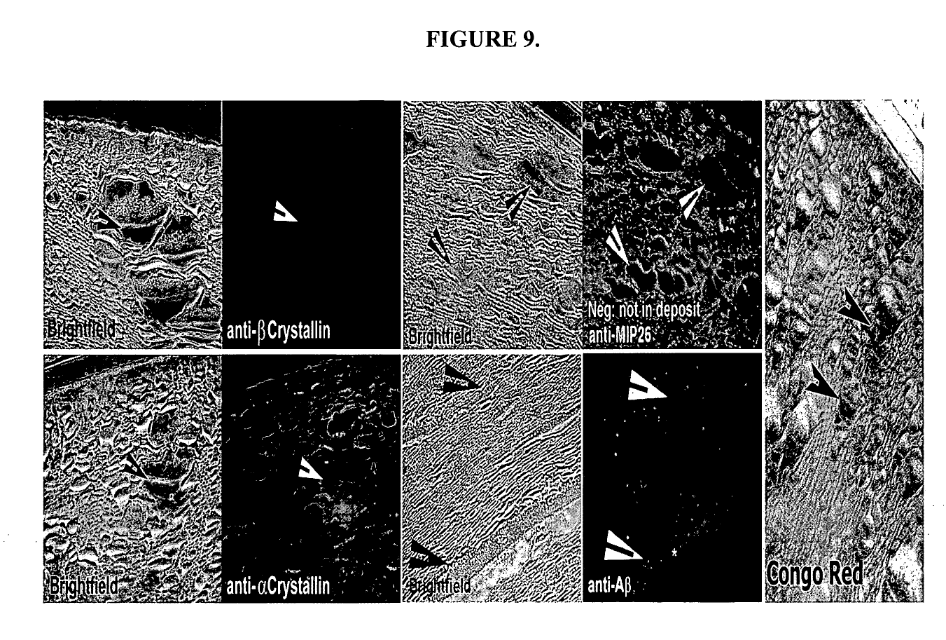

FIG. 9 demonstrates lens fiber cell degeneration in 14 month old hAPP mice. Strong fiber cell degeneration, swollen fibers, and deposits at this stage are indicated by arrows in FIG. 9. In FIG. 9 we probed fiber cells in situ with anti-Crystallin and anti-Aβ antibodies indicating corresponding and increased distribution of crystallins and Aβ in swollen fibers. This was also identified using Congo Red amyloid stain (FIG. 9), similar to Aβ-crystallin interactions indicated in late stage human lens and Tg2576 lens pathology. In contrast, Aquaporin 0 /MIP26, a major membrane protein in lenses, remains distributed in membranes and does not co-localize with crystallins or Aβ (FIG. 9); negative controls produced no signal (not shown). Arrows note dense plaques in cataract not well penetrated by Congo Red (far right panel).

Further, our studies found Aβ and crystallin deposits present in later disease stages, not readily observed at earlier stages. Examination of lenses at 1 yr. or earlier demonstrates disorganized lens fiber cell arrays (FIG. 8B) and numerous intracellular membrane vesicles. Fiber cell vesicle formation is also characteristic of human cataract (FIG. 8C, upper panel; 1), and is also seen in neurons overexpressing APP (FIGS. 8C lower panels & 8D) and seen in neurons overexpressing kinesins. Thus, lens vesicle accumulation was reminiscent of increased vesicles and lateral boutons in neurons over-expressing APP. These original observations in neurons by others that increased APP or kinesin expression gave similar vesicle trafficking vesicle phenotypes led to the understanding that the normal role of APP, acting with kinesins, is to link synaptic and cargo vesicles to the cytoskeleton for cargo trafficking and synaptic signaling in neurotransmitter release. Thus it appears that in lens as well, APP linked with Aβ formation lies at a “crossroad of cell biology and provides an Achilles heal in disease”. Disruption of cargo and synaptic vesicle trafficking is now recognized as an important early deleterious effect in AD, however its relationship is to Aβ pathology is not yet well understood. Nonetheless, these studies provide critical evidence that these fundamental cell biology processes that underlie AD pathophysiology and pathology in neurons appear to be extensively shared in lens fiber cells.

To examine in greater detail the extent to which these critical aspects of APP biology are shared in lens, and thus provides a stronger basis for understanding why shared Alzheimer pathology occurs in lens and neurons, we examined lenses for AβPP ‘neuron-specific’ synaptic vesicle trafficking partners.

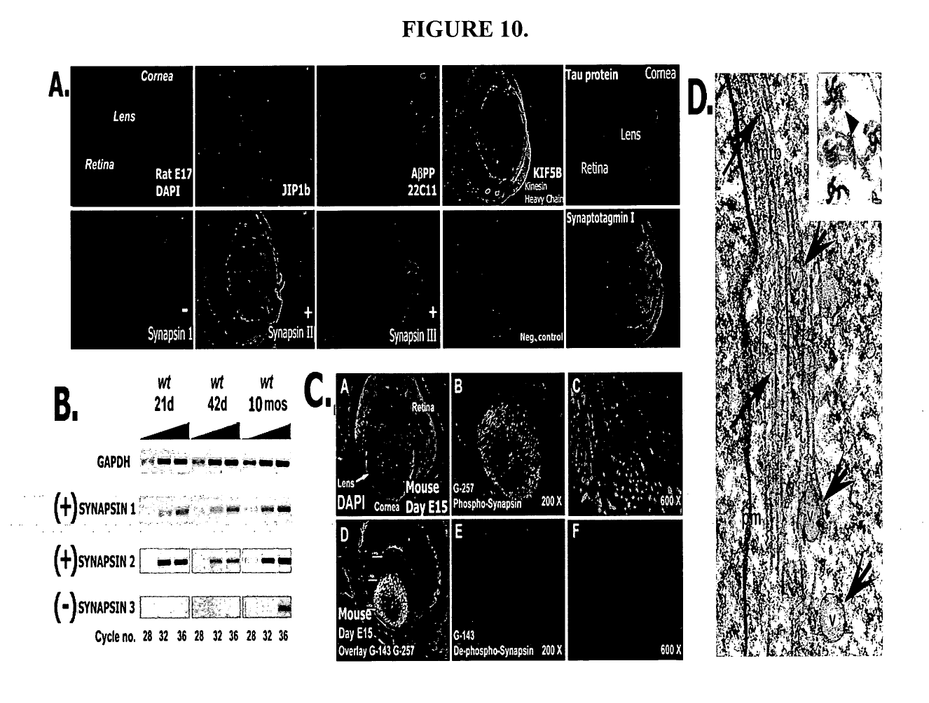

We demonstrated that a substantial complement of previously designated neuron-specific kinesins, synaptic vesicle proteins and synapsins are expressed in the lens beginning early in lens embryonic development, and continuing in the adult. FIG. 10A demonstrates overlapping coordinated expression of APP, JIP1b, kinesin motor proteins, as well as ‘neuron-specific’ synapsins and synaptotagmin I, synaptophysin, and Tau microtubule protein along the axial length of elongating fiber in fetal development and adult. To proceed further, we demonstrated neuron-like differentiation-dependent expression of Synapsin III but not Synapsin I (FIG. 10A) early in fiber cell development as fibers rapidly elongate. Correspondingly, Synapsin I with little Synapsin III, (FIG. 10B) is expressed during adult fiber cell differentiation and maturation, directly corresponding to study of neuronal elongation and maturation in vitro and in vivo.

To examine highly neuron-specific regulatory processes, we used conserved Ser-9 (Site-1) anti-Synapsin phospho-state specific antibodies (gift P. Greengard, Rockefeller, NY). Site-1 Synapsin phosphorylation fundamentally regulates Synapsin-covered vesicle neurotransmitter release at terminal ends, synapsin-mediated actin, polymerization, and Synapsin-regulated initiation of neuronal differentiation in vitro and in vivo. FIG. 10C demonstrates discrete differential distribution of Phospho- vs. De-Phospho-Synapsin proteins in E15 mouse (and E17 rat, not shown) lenses. FIG. 10C (panels E,F) are consistent with De-Phospho-Synapsin covered vesicles at apical lens fiber cell surfaces ready for regulated release. FIG. 10C (B,C) indicate Phospho-Synapsins at the lens fiber cell “soma” ready to add to vesicles for transport to distal sites, analogous to synaptic vesicle transport.

Neuron-like arrangement of microtubules in lens. An important study by Lo et al (1975) strongly complementing our findings demonstrated neuron-like microtubules along the axial length of lens fiber cells with adjacent membrane vesicles (FIG. 10D). Further the upper inset in FIG. 10D shows end-on microtubules with pinwheels that in neurons indicate anterograde/retrograde transport.

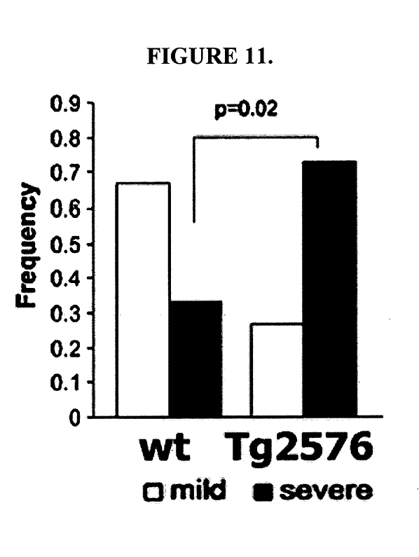

Senile cataract specifically increased in Tg2576 lens. Wolf and co-workers characterized cataract in wt mice predominantly by 25 months. In contrast, Melov et al. document Tg2576 lenses in the same genetic background (FIG. 11) develop cataract significantly earlier (300-400 days) with increased frequency. We note comparison of mouse and human APP by Hsiao et al. indicated behavioral deficits occur in mouse APP Tg mice also at later ages. In Tg2576 mice accelerated production of Aβ and plaque formation occurs by 14 months. FIG. 6C provides an example of cataract in Tg2576 mouse lens and clear control lenses similar to clear control lenses in our preliminary studies of rabbit below can be viewed in Wolf et al. It was recently reported on that lenticular and supranuclear early onset cataract in Tg2576 mouse lenses correlate with Aβ aggregates. Further, evidence indicates dynamic light scattering (DLS), also currently developed for cataract diagnosis, can be used to detect Aβ aggregates. However, Ansari and co-workers, reported lens particles with dimensions of tens of microns in diameter represent the lower limit for detection, and we note lens cells are ˜10 microns width or less, indicating cell pathology may also be present, and also consistent with detection of later stages of pathology.

Also amyloid dye similar to Congo Red detects stacked β-sheet ordered structure consistent with Alzheimer Aβ pathology in Tg2576 lenses, showing sizable aggregates in lenses permeated with the compound Me-X04 (W. Klunk, Pittsburgh, Pa.). Previously, we demonstrated amyloid dye binding can occur in normal lens which is normally highly comprised of extensively dehydrated β-sheet protein, indicated by Raman spectroscopy studies, can contribute to detection of amyloid protein structure and thus may also detect crystallins specifically. Polarized Raman studies demonstrate that lens β-sheet arrays are organized in parallel arrays, similar to classic amyloid protein structure, suggesting lens secondary protein structure has potential to contribute to background amyloid β-sheet signal.

Human genetics also links senile cataract with presenilin loci, similar to AD. Gene mapping links congenital and age-related cataract with Presenilin-1 and -2 loci. Three studies mapped familial and age-related cataracts to 1p36 (near PSEN-2). The latter study states this site overlaps with congenital autosomal dominant cataract of the ‘Volkman’ type and with autosomal dominant posterior polar cataract. Added significance is seen in association between cataract and PSEN-1 on 14q24. Anterior polar cataract represents 3%-14% of congenital cataracts, and maps to 14q24. Moross identified a chromosome 2-14 translocation associated with cataract, and transcript analysis demonstrates PSEN-1 is likely affected.

Vision defects specifically related to cataract occur in up to 50% of AD affected individuals, and represent an important and under-investigated complaint that appears early-on in AD. Vision defects are a leading first complaint causing AD affected individuals to first seek out a physician. Previously, investigators trying to understand what the nature of vision defects might be, often pre-supposed these defects to be due to changes in the brain visual cortex in AD. However, this is not borne out, and upon careful analysis lens pathology in AD patients was demonstrated to be independent of cognitive effects. Moreover, findings of visual defects in AD led to investigation of the retina & optic nerve and demonstrated these tissues remain unchanged in AD, particularly early-on. We note that, like cataract, low visual acuity and contrast sensitivity also occurs in DS, and agree with early-onset cataract and early-onset AD that occurs in DS, for which we provided further evidence in our study of hAPP Tg mice.

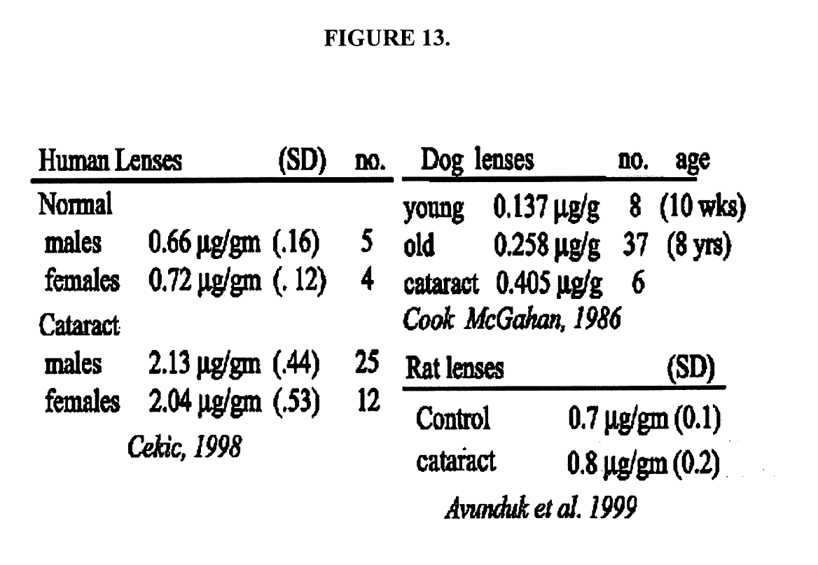

Cu increase in lens is a well-characterized & consistent biomarker of lens aging and increases further in cataracts; and Aβ is key factor in lens Cu accumulation. Atomic absorption studies over the last 20 yrs document Cu increases (but not Zn) in mammalian lenses during aging, and much more so in cataract formation (FIG. 13), in lenses that express high Cu-affinity Aβ peptides. However, in light of studies indicating Cu readily replaces Zn in Aβ complexes, decreased Zn in rat cataract would appear to have a similar effect. Preliminary experiments below indicate Aβ is a key factor in lens Cu accumulation, consistent with ELISA detection of increased Aβ and colorimetric detection of increased Cu. Our preliminary Raman spectroscopy analysis of normal and cataractous lenses below identifies increased Aβ-metal complex increase in cataracts.

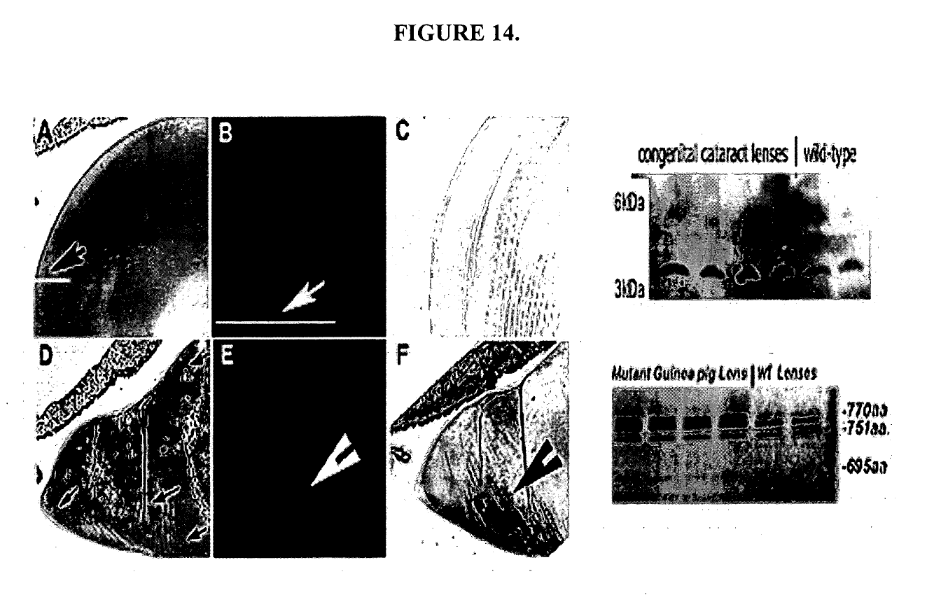

Transgenic expression of high Cu affinity human Aβ peptides in mouse lenses significantly increase Cu detected in 1 yr-old hAPP mouse lenses. Assays of Cu (FIG. 13) together with analysis of Aβ peptide sequences (FIG. 2) strongly indicate that Aβ metal chelating amino acids have a key role in determining Aβ metal affinity:and as a result have a key role in determining the nature of Aβ pathology in lens, related to their influence on AD Aβ pathology. To test these ideas further we used well-characterized in situ Cu histochemistry methods to probe mouse lens histological sections. Using these methods (similar to FIG. 14), we readily detected Cu in lens in our YAC hAPP transgenic mouse model, primarily in outer cortical lens fibers, and little Cu staining was detected in wt lenses of the same genetic background (not shown). hAPP mice express physiological levels of human APP, and for example also retain native alternative splicing patterns that we demonstrated in human lenses. This cortical distribution is also consistent with our earlier studies of APP and Aβ expression in rat, monkey, and human lenses. In addition the same Cu distribution was observed with Timm histochemistry (not shown). When HCl pre-leaching was included to remove Zn and Fe metals, this still produced no apparent difference in Cu distribution.

We note that wt and Tg hAPP mouse lenses each express a normal complement of lens proteins, further indicating a specific role for human-like Aβ peptides in lens Cu accumulation. Although we understand these studies do not rule out an unspecified effect on lens, or systemic effects on metal homeostasis contributing to lens Cu accumulation, the data are consistent with findings in human, dog, guinea pig, and rabbit vs. rat and mouse and the high Cu affinity Aβ peptides in those mammals.

We next tested this hypothesis in Tg2576 mouse lenses (gift, G. Pasinetti, Mount Sinai, N.Y.). Using sandwich ELISA assays kits (Biosource Interantional/Invitrogen, Carlsbad Calif.) we readily detected Aβ₁₋₄₀ and at ˜3-4 fold lower levels Aβ₁₋₄₂ in 1 year-old Tg2576 mouse lenses. As expected, we obtained no signal in wt lenses (data not shown). However, Cu measured in these lenses using Quantichrom colorimetric Cu kits (BioAssay System, Hayward, Calif.) demonstrated significantly higher Cu in Tg2576 lenses and was not observed in wt lenses (not shown).

Aβ peptides are a key element in local Cu distribution in lens. In brain, Cu and Zn co-localize with focal deposits of Aβ peptides. Most recently, Miller et al. used infrared and X-ray imaging to demonstrate this point that focal accumulation of Cu and Zn co-localizes with Aβ deposits in senile plaque deposits in Alzheimer's disease brain tissue agreeing with co-localization data above. To examine Aβ Cu co-localization further and the role of Aβ peptides in the accumulation and distribution of Cu in lenses, we examined the distribution of Aβ peptides and Cu that we detected in guinea pig lenses in a congenital cataract model guinea pig strain. As noted in FIG. 2, guinea pigs express human-like APP protein and high Cu affinity Aβ peptides. In lens histological sections shown in FIG. 14, lens regions in congenital cataract mutant guinea pig lenses were probed using Timm stain histochemistry similar to mouse lens experiments described above. The dark stained region in lens sections in FIG. 14F identifies increased distribution of Cu in mutant lens only that overlaps with regions of increased Aβ peptides detected by immunohistochemistry in adjacent lens sections FIG. 14E (not in wt). In these congenital cataract lenses, the anterior-posterior fiber cell length is considerably shortened. Aβ peptides detected using anti-Aβ Mab6E10 produced a similar pattern in adjacent sections using a 4G8 monoclonal preparation and consistent negative controls. Our analysis of Cu using Timm histochemistry again included Zn—Fe pre-leaching HCl steps and detected no changes in Cu distribution, consistent with analysis of metals in normal and diseased lenses using atomic absorption in mammalian lens.

Cataract in this model in large part is due to increased oxidative stress. To confirm APP and Aβ expression in guinea pig lenses in wt and mutant lenses, the top right panel in FIG. 14 shows increased immunohistochemical Aβ detection in mutant lenses. In the bottom right panel, RT-PCR analysis of APP transcripts in mutant and wt lenses identifies moderately increased in APP gene expression in mutant lenses suggesting increase Aβ precursor substrate is present during development. However, no significant differences in any of the alternative splice products are apparent. Similar to humans and mice, APP variants containing exon 7±8 predominate. In addition, it is speculated shorter fiber cell length may also relate to APP overexpression contributing to cargo vesicle transport defects.

Systemic oxidative stress due to Thiamine (Vitamin B1) deprivation is consistent with our shared biological diathesis model that lens and brain have shared underlying genetic vulnerabilities to stress contributing to Alzheimer protein pathology in both tissues. Thiamine as TPP permits fermentative production of reducing power particularly needed in cells with high surface to volume ratios like lens fibers and neurons. APP pathology is documented in brain using this physiological regimen described by Gibson and co-workers, and we demonstrated corresponding APP pathology predominantly in posterior fiber cell degeneration with locally increased APP & Aβ. We note, Cu & Zn were not investigated.

Laser-based Confocal Raman spectroscopy for detection of Aβ-metal complexes in lens. Methods used to date for detecting and measuring Aβ in lens or brain predominantly focus on antibody-based detection of isolated protein samples and include ELISA and immunoblot antibody assays, as well as immunoprecipitation procedures. For the detection of Cu and other metals atomic absorption spectrophotometry as well as colorimetric methods are most often used. Since the transparent lens is amenable to light-based detection methods and spectroscopy, and provides an easily accessible route for positioning detection equipment, we proposed that Raman spectroscopy which uses newly designed portable optic fiber probes can provide an ideal method for detecting and characterizing Aβ-metal complexes in the lens. Raman spectroscopy already has a track record for use in the eye that can facilitate its development for monitoring lens Aβ-metal biomarkers for: 1) cataract and 2) AD diagnosis. Raman spectroscopy is now used to measure antibiotics and also glucose in the anterior segment in front of the lens in diabetic patients, and is in use for measuring pigments in the retina located behind the lens.

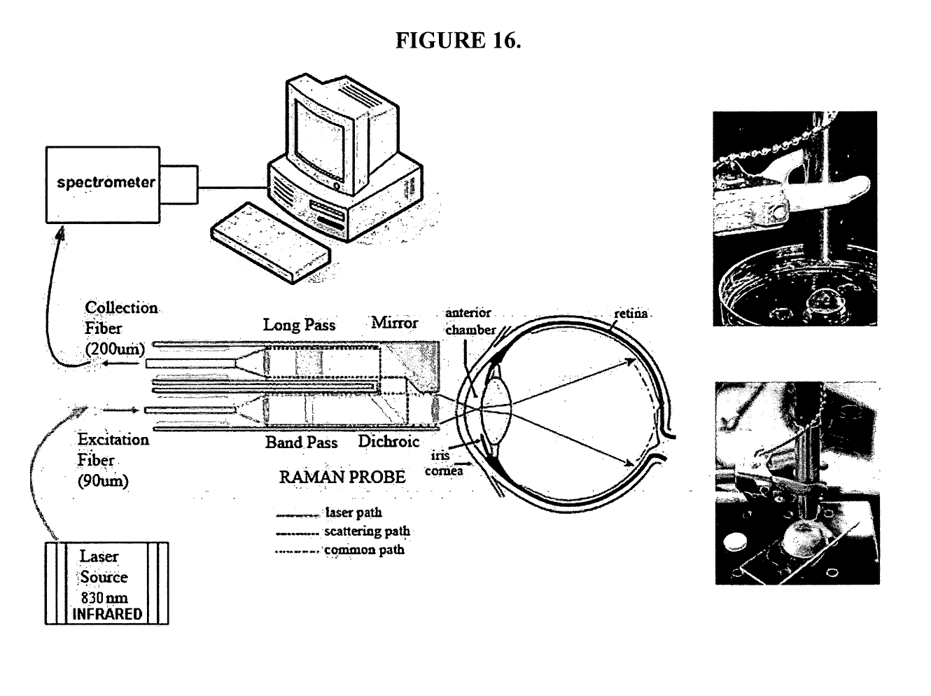

FIG. 16 diagrams the apparatus we are using to obtain Raman spectra of lenses. Currently, we obtain spectra from isolated lenses or dissected anterior region of the eye, however, whole globes are also used. At this stage we use isolated lenses to allow easy positioning to probe and rapidly assess all lens regions. This key development in our research was instrumental in our choice of available models of cataract and. AD pathophysiology enabling us to use an animal model with large lenses amenable to regional analysis and similar in shape to human lens.

Physiological Animal model of high dietary cholesterol and added trace Cu to drinking water. Here, we have chosen to examine a physiological model of systemic pathophysiology in rabbits. This model based on high dietary cholesterol in rabbits has been extensively characterized in its ability to produce characteristic AD brain pathology and behavioral deficits. Cholesterol is closely linked with AD and its mechanism, and with cataract in epidemiological studies. High dietary cholesterol has been used previously to produce cataract. High LDL (“bad”) serum cholesterol is strongly associated with cataract and AD. Cholesterol is also a critical factor in diabetic cataract with diabetes being increasingly viewed as a risk factor for AD and cataract. Further, high cholesterol produces lens opacities and strongly augments cataract formation in streptozotocin-induced models of diabetes. In individuals with high cholesterol, cortical lens opacification is the most prevalent sign of dyslipidemia linked with disrupted cholesterol metabolism. Abundant biochemical, epidemiological, clinical, and animal model data demonstrates cholesterol has a key role in AD. High cholesterol also profoundly affects development of amyloid pathology in Tg AD models. Hypercholesterolemia accelerates Aβ pathology, and conversely cholesterol-lowering drugs reduces Aβ pathology in an AD transgenic mouse model based on Tg2576. However, it appears highly significant that the Tg2576 heterologous Prion protein promoter used in this model, in work from our previous study of PrP expression in lens and its relationship to cataract, is significantly stimulated by oxidative stress. In addition, there is abundant data linking statin use with decreased AD risk. Although statin use has not been associated with lens, in organ culture, statins affect lens isoprenylation of small GTPases. The high cholesterol model in rabbits was found to reproduce over 12 hallmark features in AD.

High dietary cholesterol and trace Cu bioavailability have synergistic effects on AD brain pathology. Trace Cu added to drinking water in high cholesterol fed rabbits significantly increases AD brain pathology and behavioral deficits. In addition, biochemical mechanisms have been identified that are consistent with synergistic action of Cholesterol and Cu. For example, Cu added to cells increases APP expression, and increases cholesterologenic gene expression acting through Cu-response promoter elements. Conversely, Cu depletion down-regulates APP production, Cholesterol has also been identified as a key substrate for oxidation by Aβ/Cu that mimics cholesterol oxidase enzymes and produces H₂O₂. The apolipoprotein E type 4 (apoE4) allele, which encodes a major cholesterol transporter in blood plasma and the brain, represents a strong genetic risk factor for both familial and sporadic AD. It has often been noted neurons have very high membrane cholesterol levels, where APP resides. However, lens fibers are considered to have the highest membrane cholesterol content in nature, and examination of lenses demonstrates cholesterol oxides are significantly increased in cataracts. Together, biochemical and epidemiological evidence strongly link cholesterol with AD as well as cataract. Regarding the role of Cu, the National Tap Water Database report (www.ewg.org/tapwater/) found that >78 million in the US drink Cu contaminated water, and the EPA reports information on its website that common pipe corrosion is key in random Cu contamination, and highlight cholesterol & Cu further as an AD health risk in the US and abroad warranting more research. We note that the EPA-accepted level for human drinking water is 1.3 ppm vs. 0.12 ppm in the present animal model.

Below are the findings from a 10 wk pilot study with evidence for the following: (1) Cataract occurs in lenses from high cholesterol/trace Cu rabbits examined at 10 wks; (2) Aβ₁₋₄₀ and Aβ₁₋₄₂ peptides increase significantly in high cholesterol/Cu rabbits; (3) Copper increases more than 2-fold in high cholesterol/Cu fed rabbit lenses at 10 wks; (4) Raman spectroscopy demonstrates specific increases of Aβ-metal complexes consistent with Aβ and Cu increases indicating this model is also appropriate for proposed characterization for diagnosing cataract and monitoring corresponding AD pathology in AD patients.

Pilot Study (10 wks):

Control Rabbits: Normal Rabbit Chow/dH2O Drinking Water

Experimental: Normal Chow+2% Cholesterol/dH2O+0.12 ppm Cu

Analysis of Aβ peptides and Cu in high cholesterol fed rabbits with trace Cu added to drinking water. To characterize the development of Raman spectroscopy in the lens, one group of rabbits was placed on a 2% added cholesterol Purina chow diet and given 0.12 ppm Cu added to purified dH₂O drinking water for 10 wks. A second group of rabbits was given standard Purina rabbit chow and purified dH₂O drinking water for 10 wks. This regimen followed a protocol described by Sparks and co-workers in several recent publications cited above. The animals were handled according to our animal protocol following approved practices for laboratory animals. Eyes were removed and taken for analysis of lens Aβ peptide levels and for Cu content.

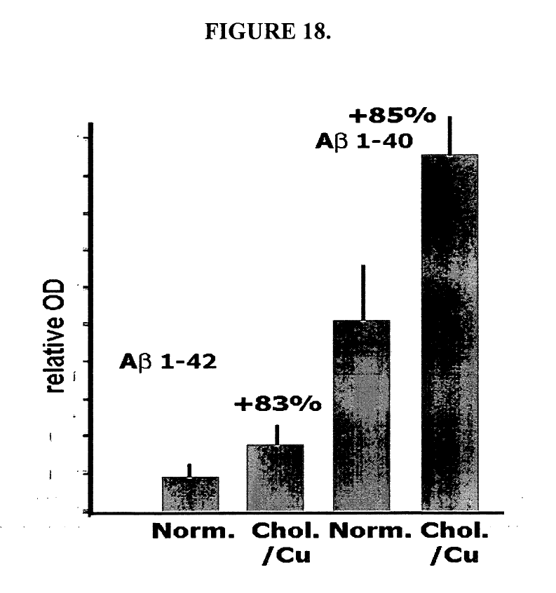

Aβpeptides increased significantly in high cholesterol/high Cu fed rabbit lenses after 10 wks. To analyze Aβ production in lenses, we used sandwich ELISA assay kits (Biosource/Invitrogen, Carlsbad Calif.) specific for analysis of Aβ1-40 and Aβ₁₋₄₂ peptides. Lens solubilized in Guanidine HCl with 50 mM Tris pH 8.0 were used in each assay. After addition of detecting antibodies and appropriate incubations, a colorimetric spectrophotometer was used to determine the amount of antibody linked enzyme reaction product corresponding to Aβ peptides present in the sample. Results in FIG. 18 demonstrate an approximate 85% increase in Aβ₁₋₄₀ and Aβ₁₋₄₂ peptides in lenses from high cholesterol/high Cu diet rabbits compared to controls. These results from lenses from four control and four experimental rabbits show Aβ₁₋₄₀ is approximately 3-fold higher than Aβ₁₋₄₂ in control and high cholesterol diet/Cu lenses. These results agree with lens Cu assays and Raman Spectra analysis presented below.

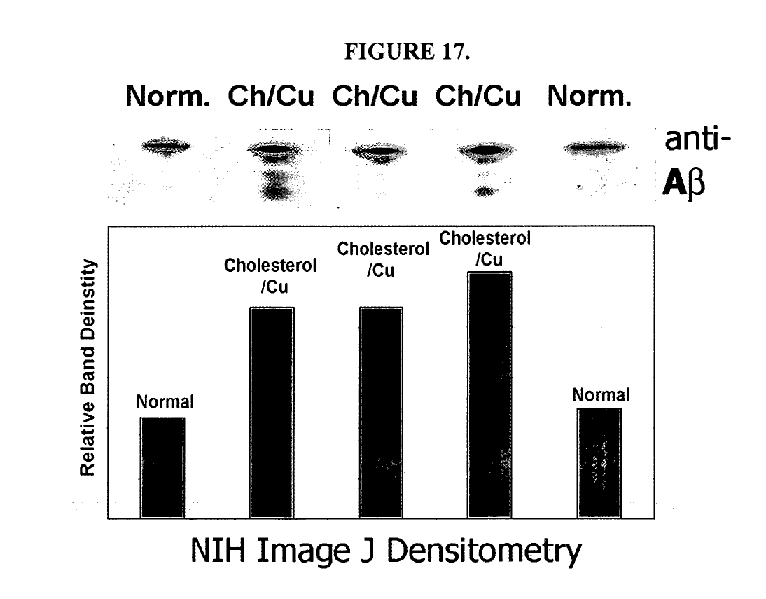

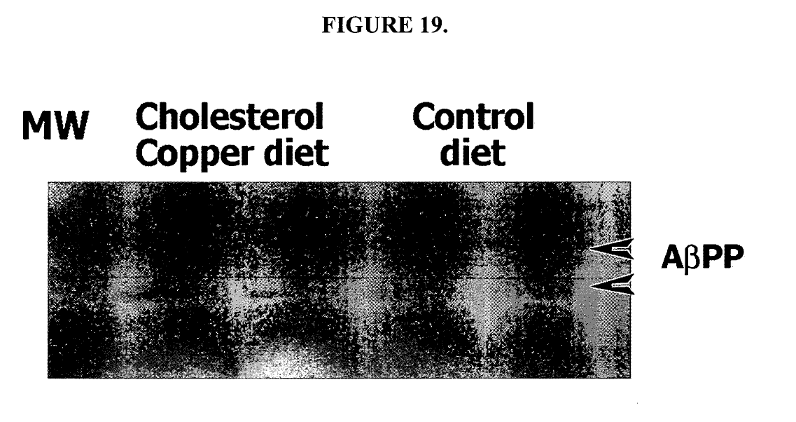

Immunoblot detection of lens Aβ peptides indicates a similar increase in Aβ peptides in high cholesterol/Cu fed rabbit lenses. FIG. 17 demonstrates western blot analysis of Aβ peptides in rabbit lenses consistent with results from sandwich ELISA assays. Western blots permit analysis of substantially greater amounts of protein. Protein samples from control and experimental lenses resolved on Tricine gels (Novex) and electroblotted to filters were probed with anti-Aβ peptide monoclonal antibody (4G8), and detected with secondary antibodies and horseradish peroxidase enzyme substrate detection. Adjacent bands detected in lens protein samples from normal diet fed rabbits run next to lens samples from high cholesterol/cu fed rabbits were also quantified using NIH Image (NIH image J for PC) shown below. In FIG. 19 our preliminary examination of APP expression in two high cholesterol/Cu fed rabbit lenses, and two control lenses detected a slight increase in APP parent protein using mouse monoclonal 22C11 anti-APP antibody in the immunoblot assay, and will be followed up using related antibody preparations and techniques. This antibody may detect APP homologues.

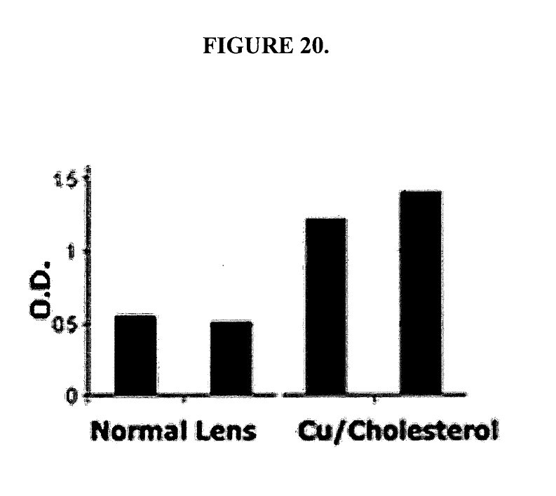

Lens Cu increases ˜2.5 fold in high cholesterol/Cu lenses, and consistent with increased lens Aβ and its proposed role in lens Cu accumulation and consistent with Raman spectra below. An examination of Cu levels in rabbit lenses in our pilot study shown in FIG. 20. Using colorimetric assays of Cu (Quantichrom Cu assay kit, BioAssay Systems, Hayward Calif.) in experimental and control lenses identified a significant increase in Cu in high cholesterol/Cu fed rabbits. Product monitored at OD at 354 nm is a direct measure of Cu in lens samples. Cu in control and high cholesterol/Cu fed rabbits is shown in from two experiments using 2 normal lenses and 2 experimental lenses each.

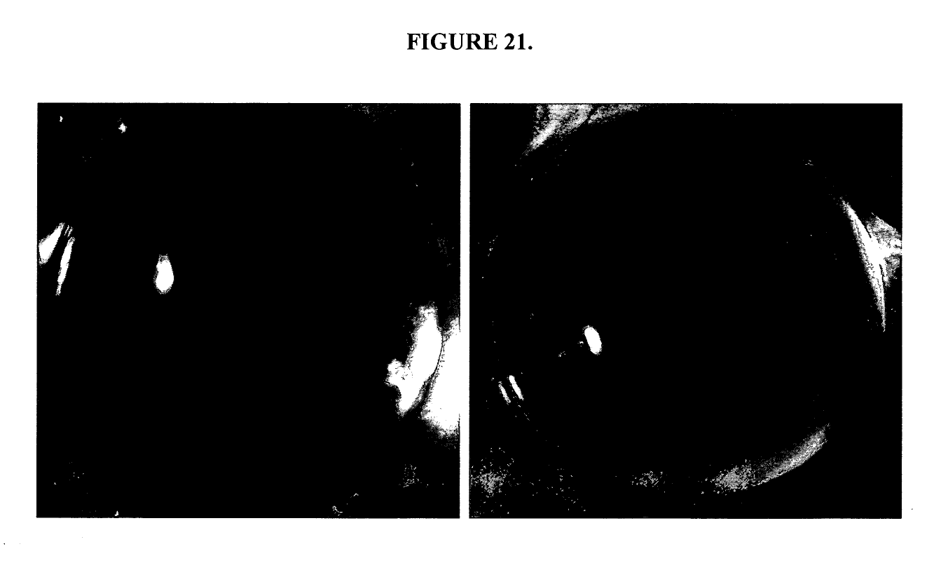

Cataract is produced in lenses from rabbits on the high cholesterol/Cu regimen. FIG. 21 shows an example representative of lens opacification (cataract) present in cholesterol /Cu fed rabbit lenses after 10 wks. Lenses are photographed on a dark background and reveal light scattering in experimental lenses not seen in controls.

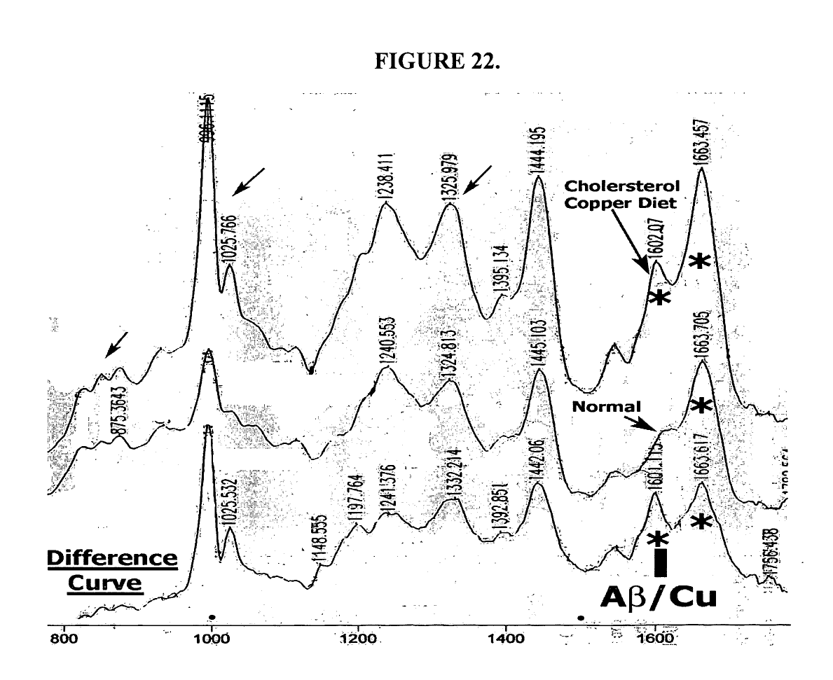

Confocal Laser Raman Spectroscopy can be effectively used to measure early signature changes in lens Aβ and Cu at the molecular level corresponding with cataract formation in a well-characterized AD animal model. The Raman setup diagramed in FIG. 16 illustrates one embodiment of a Raman spectroscopic device for use in the methods of the invention. The setup in FIG. 16 uses a portable hand-held laser Raman probe and portable spectrometer to assay chemical structures in intact rabbit lenses. In FIG. 22 two complete Raman spectra are graphed one above the other. The upper Raman spectra trace is from a high cholesterol/Cu rabbit lens and below that from a normal diet fed rabbit lens. The region of the spectra shown is from 800-1700 cm¹. The probe assays a volume approximately 0.3 mm³ and the ˜8 mos-old rabbit lenses is approximately 1 cm in diameter. This also allowed us to obtain spectra from different lens regions at the perimeter and the lens center, and at different depths in the lens. Comparison of Raman spectra from rabbit lenses at the ˜1604 cm⁻¹ signature of Cu N-tau His interactions with Aβ peptides are remarkably consistent between experimental lenses and different from controls and consistent with published spectra from 20 or more years ago (blue arrows).

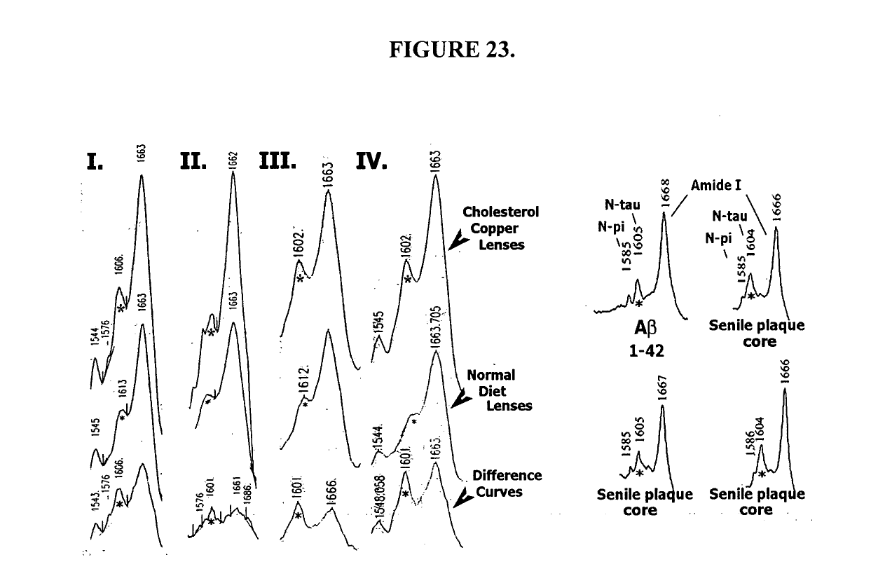

Raman analysis of cholesterol/Cu rabbit lenses produced spectra strikingly similar to spectra from human AD brain plaque cores & in vitro study of Aβ/Cu interactions, not present in normal rabbit lenses. The left panel of FIG. 23 below marked I-IV shows spectra in the region from 1500-1700 cm⁻¹ from high cholesterol/Cu fed rabbits and control diet rabbits. Lenses are arbitrarily paired together, and computed difference spectra from high cholesterol/Cu vs. normal lenses are shown in the traces below using a constant reference. Raman scans for the high cholesterol/Cu rabbit lens produced strong signature peaks at ^{˜}602 nm indicating Aβ-Cu (or Zn) inter-molecular interactions that were considerably higher than in spectra from control lenses (see difference curves). High cholesterol/Cu lens in FIG. 23 produced minor shoulder peaks at ˜1585 cm⁻¹ indicative of intra-molecular Aβ/Cu interaction. Previously, other labs determined in vitro that Raman band intensities at ˜1604 and ˜1585 identify discrete signature changes due to specific Aβ peptide interactions with Cu or Zn. These data are also completely consistent with Aβ and Cu increases measured in biochemical assay and Cu analysis. Together, those findings suggest that Aβ-metal complexes we detect largely involve Aβ-Cu. For comparison, the right panels in FIG. 23 demonstrate Raman spectra of Aβ-metal interaction in vitro using material from AD brain senile plaque core material from three confirmed AD cases. Remarkably, spectra obtained for four lenses from cholesterol cataract lenses and from senile plaque (SP) cores isolated from AD brain are essentially identical.

Conclusions. Strictly non-invasive and non-destructive Raman spectroscopy methods have significant potential for measuring increases in signature peaks that identify specific Aβ/metal complexes during cataractogenesis. Raman signature intensities at 1604 cm⁻¹ and 1585 cm⁻¹ agree with increases in vitro assay of Aβ & Cu. Also, Aβ/metal signature peaks in cholesterol/Cu lens spectra are consistent with Aβ/Cu or Zn interactions at imidazole Nitrogens. These data suggest small Aβ oligomers may be detected in early stages of AD pathology. In contrast, amyloid detection similar to amyloid dye studies by LeVine determined that dye binding and diagnostic fluorescence shifts require extensive amyloid structures in order to be detected. However, signature Raman peaks at ˜1602 nm are also consistent with Aβ fibrils and extensive aggregates.

These data indicate that Raman methods can detect biomarker changes in Aβ/Cu pathology at early disease stages. Amide I β-sheet intensities are present in control lenses as expected, also seen in previous lens Raman studies by others. Aβ/Cu peaks are not affected by extensive normal lens β-sheet^{43b} amyloid-like structure.

Experimental Design and Methods

Aβ analysis. Sandwich ELISA kits from Biosource/Invitrogen (Carlsbad, Calif.), (also Sigma, St. Louis Mo.) are used to detect Aβ₁₋₄₀ and Aβ₁₋₄₂ peptides. Briefly, this procedure involves solubiltization of tissue in 8 volumes of cold 5M Guanidine HCL in a 50 mM Tris buffer. This buffer is compatible with Quantichrom kit colorimetric metal assays described below, and each lens (each consistently ˜300 mg in prior analyses) provides more than sufficient material for Aβ ELISA and colorimetric assays. The strongly denaturing solution effectively defeats potential sources of protein degradation, and allows samples to be stored at ˜80° C. for long periods a freezing and thawing (for Aβ detection according to the manufacturer). Further dilutions or dialysis for PAGE study includes protease inhibitor cocktails. Separate kits for Aβ₁₋₄₀ and Aβ₁₋₄₂ contain Aβ standards, antibodies and buffers. A microtiter plate is provided with pre-adsorbed pan-specific anti-Aβ antibody. Aliquots of solubilized and denatured samples are added, followed by affinity purified antibody specifically for kit-specific Aβ₁₋₄₀ and Aβ₁₋₄₂ peptides, determined by the variable C-terminus. After incubation and washing, a 2° antibody conjugated to horseradish peroxidase is added, followed by substrate. Fluorescent and colorimetric kits are available and departmental plate readers for both are available. Standard curve preparation and replicate controls are discussed in detail by the manufacturer in literature provide with references and examples, and experiments to confirm peptides detected are described below.

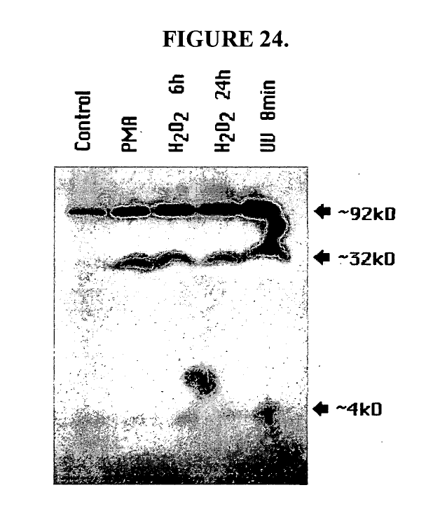

APP expression. APP expression levels can impact Aβ peptide formation, and for example is proposed as a factor in gene dosage effects contributing to early-onset AD (and early cataract, 44) in Down syndrome. Our study of monkey and rat lens organ cultures exposed to oxidative stress identified increases for both APP and Aβ peptides on western blots and by IHC in situ (FIG. 24;43). Increase in APP increases substrate availability for secretases, and impact amyloidogenic vs. non-amyloidogenic processing. In addition, APP levels relative to other transport motor partners can participate in altered cargo vesicle transport cell biology at very early stages in human AD and Tg mouse models. To measure APP protein levels APP ELISA kits (Biosource/Invitrogen, Carlsbad Calif.) are used to examine APP expression in rabbit lenses. Here, lenses are solubilized in10 mM Tris, pH 7.4, 100 mM NaCl, 1 mM EDTA, 1 mM EGTA, 1 mM NaF, 20 mM Na4P2O7, 2 mM Na3VO4, 1% Triton X-100, 10% glycerol, 0.1% SDS, 0.5% deoxycholate, 1 mM PMSF and Protease inhibitor cocktail (Sigma). This tissue solubilization procedure is compatible with PAGE electrophoresis and immunoblot analysis. Alternatively, samples are dialyzed against 7MUrea/1% SDS to increase solubility if required.

Immunoblot & immunoprecipitation. To confirm peptide and proteins detected in ELISA assays, lens samples are analyzed on immunoblots using Tris or Tricine buffer based PAGE systems geared to resolution of particular molecular weight ranges (Novex-Invitrogen and BioRad). Similar to preliminary studies, proteins resolved by molecular weight and blotted to filters are probed with antibodies against Aβ. We have successfully used 6E10, 4G8 monoclonal antibodies, and affinity purified rabbit anti-Aβ (Zymed-Invitrogen). To detect APP we use monoclonal 22C11, and polyclonal C-terminal specific (Zymed). Several factors including anitibody preparation contribute to successful resolution and detection of proteins. The lens is unique due to high expression of specific crystallin proteins. In some cases to detect peptides on filters, it may be necessary to boil membranes in PBS to enhance antibody detection. A further method to confirm our identification of APP and Aβ proteins will use immunoprecipitation of protein extracts followed by immunblot detection with related antibody preparation.

Mass spectrometry. To specifically confirm the presence of Aβ peptides at the molecular level, we will follow published protocols. We will use 4G8 or 6E10 monoclonal antibodies to precipitate Aβ peptides. This is followed by Protein G/A-agarose to pull-down complexes for application to the mass spectrometer. Spectra are measured with Applied Biosystems or Voyager Maldi-TOF systems. Aβ₁₋₄₀ and Aβ₁₋₄₂ peptide specific antisera are also available and are used for comparison.

Metal Analysis. A key goal is to quantify levels and accumulation of Cu, Zn and other metals in lenses and relate this to Aβ accumulation and Aβ-complex formation. We use two in vitro metal detection methods, and Raman spectroscopy to detect and characterize Aβ-metal complexes in situ in lens. To date, spectrophotometric analysis has also only been used in vitro.

Colorimetric assays of metals. Cu and Zn analyzed in lens samples using Quanichrom Assay kits in lenses. Kits each provide standards and reagents. Metal detection kit sensitivity is 8 ug/dl for copper and the manufacturer indicates Zn assays are in the same range. We note many previous atomic absorption spectrophotometry analysis of lens demonstrate Zn is present at higher levels in mammalian lenses. As noted above kit assays are compatible with extraction buffers described in ELISA assays section, and not affected by moderate levels of guanidine or Urea in buffers.

Atomic absorption methods. Atomic absorption analysis of Cu and other metals in lens samples is carried out. Cu and other trace elements in tissue samples are analyzed by electrothermal atomic absorption spectrometry. Our preliminary colorimetric analysis, as well as published atomic absorption spectrometry studies of rabbit lenses indicate Cu and Zn are present at high enough concentrations in normal lenses, and increase in cataract and can be measured here. Lenses is ashed using a low temperature asher that uses a radio frequency field to generate singlet oxygen so that the samples can be ashed without the possible oxidation at high temperature or by concentrated oxidizing acids. This will also prevent any loss of metals that may occur at high temperatures and will help prevent detection of a “blank” high for copper or other metals, since trace elements can be found in acids. The ashed residue is dissolved in very high quality 1% nitric acid and analyzed using appropriate blanks, standards, and standard reference materials. The sensitivity of graphite furnace atomic absorption spectrometry for Cu is about 5-20 pg for 1% absorption (0.0044 abs. units) similar to other metals and produces an easily detectable signal. However, analysis of a specific samples for Cu depend on additional factors, including the “matrix” and sample preparation on the analysis for Cu.

Serum cholesterol and Cu analysis. A primary goal is to demonstrate changes in Aβ and Cu in lens pathology, and compared with brain, However, a large number of epidemiology and biochemical studies strongly implicate cholesterol in AD and cataract. In addition, systemic Alzheimer pathophysiology is linked with serum Cu increases as well, and Cu can act as a cholesterol oxidase. To benchmark changes in serum cholesterol and to investigate changes in serum Cu, we obtain serum samples at the time rabbits are taken for tissue analysis. Cholesterol and Cu assays are sent to professional services for analysis, and an aliquot kept at −80° C. for colorimetric and for atomic absorption metal assays. Serum samples are sent for testing to Antech Diagnostis, NY or Radii Research Animal Diagnostic Laboratory, at the U. Missouri, Columbia Mo.

Raman Spectroscopy. Raman excitation wavelength in the near-infrared region is chosen to diminish biological fluorescence and minimize tissue damage. The apparatus consists of a CCD detector coupled to a spectrometer with digital output to a computer, a near-infrared laser source, and an excitation/collection Raman probe as diagramed above. The system consists of an 830-nm diode laser with an adjustable power output. We use 125 mW, power with 5 cm⁻¹ resolution, with an electronically cooled CCD array detector, and a Raman probe. Laser light passing through the probe is focused on the sample and same probe also housing the detector is used as the collection sensor. The optical system permits only signals generated near the excitation beam to be efficiently coupled into the collection fiber. This design will aid in preventing retinal damage when the system is used to collect data in vivo. These same parameters also apply to the present in vitro set up. The probe we use is confocal, with the fiber core providing the aperture and the output lens defining the focal area. In experiments where we use intact eye globes or in vivo analyses, the power to the back of the eye is spread over about half the retina because the light is focused in the lens, and this means the power per unit area is rather small. We have calculated that our beam would have a lower power per unit area than a typical fluorescent ceiling light, consistent with plans for application in patients.

Raman spectroscopy analysis of regional distribution of Aβ-metal complexes. These experiments assay Aβ complexes at different points within one intact lens. Raman studies in in vitro isolated lens protein can be significantly different from structure observed in the intact lens. In our earlier histological localization of Aβ in human cataract and cultured rat lenses exposed to stress, we identified Aβ production primarily in the lens periphery, and along the entire perimeter. To investigate this relationship in greater detail Raman spectra is obtained at four equidistant points around the lens periphery. As noted, the region assayed by the laser probe and optical sensor is approximately 0.3 mm³. Spectra are obtained at center of the lens visual axis as well and at the lens surface; then focus 1-2 mm below the surface to sample the lens interior. These studies are compatible with rabbit lenses that are ˜1 cm in circumference and ˜0.5 cm from anterior to posterior surface, similar to human lenses, This is in contrast to ˜2 mm diameter round mouse lenses.

Eye histology. To examine lens pathology and prepare lenses for immunohistochemical and histochemical analysis, eye globes removed intact are fixed in 4% paraformaldehyde in PBS pH7.4. Paraffin sections of the entire eye are examined for lens pathology in hematoxylin and eosin sections. Thioflavine and Congo Red detection of amyloid will be used as in Frederikse et al. Antibody detection using standard methods demonstrated is used to evaluate the distribution of Aβ peptides, and APP. In addition, we use in situ Cu histochemistry similar to preliminary studies on guinea pig lenses above using Timm and rhodanine Cu procedures to identify Cu distribution in lenses similar to FIG. 14. Lens fiber arrays and vesicle formation are examined analogous to our study of hAPP lenses to determine if related vesicle defects demonstrated in human and hAPP transgenic mouse lenses are present.

Characterization of lens opacification and cataracts. Lenses are photographed to document opacities and slit lamp used to monitor and characterize cataract formation using a hand held and a table top apparatus. We correlate the findings with biochemical & chemical measurements and lens histopathology. Cataracts are related to standard methods for human cataract classification using the CCRG rating criteria.

Examination of rabbit brains. Previous studies of AD brain pathology in this model primarily focused on histopathology markers (and biochemical (ELISA) measurements of Aβ peptides and demonstrated significant AD pathology in brain. Rabbit brains are removed intact for analysis. After euthanasia by approved protocols, whole eye globes are taken. Subsequently, the skull cap is removed and the intact brain is gently lifted out, and placed in liquid nitrogen for biochemical analysis or buffered 4% paraformaldehyde and fixed for two weeks for Immunohistochemical studies. Fixed brain tissue will be prepared for paraffin sectioning (˜10μm thick). This method preserves tissue morphology and is easier to stain than free-floating thick sections that can lead to tissue tearing. However, we note that this will increase the number of fields required for counting of Aβ positive neurons. Immunohistochemistry uses 6E10 & 4G8 monoclonal antibodies. Sections treated with 1% H₂O₂ to ‘kill’ endogenous peroxidase activity, are blocked in 4% Donkey serum, and incubated overnight with antibody for standard IHC detection. For analysis of amyloid deposition, sections are stained with amyloid dyes congo red & thioflavine or crystal violet. Birefringence is examined with cross-polarizing filters. Thioflavin fluorescence is examined using appropriate immunofluorescence cubes indicative of interaction with stacked β-sheet amyloid protein structure. Microscopic counts of immunoreactive neurons are taken from paraformaldehyde-fixed sections in which the number of Aβ immunoreactive cells within 10 randomly chosen 0.5×0.5-mm square fields are counted at 20× magnification in thick sections or 50 fields in thin sections, and averaged by a researcher blinded as to the treatment of the rabbits. We note that the general increase in tissue preservation and quality of morphology in thin sections, in our hands, more easily allows us to locate regions for counting. Pairwise comparisons of the average numbers of immunoreactive cells between the dH20/normal chow control rabbits and each of the separate treatment groups is first performed, followed by analysis of variance (ANOVA) with a statistical significance level of p<0.05.

Aβ peptide assays. Aβ ELISA kit (Biosource, Camarillo Calif.). Frozen cortical tissue is extracted in 8 volumes of 5 M guanidine HCL as for lenses in 50 mM Tris buffer at pH 8.0. Modifications described by Newell et al 2003 will be included if required to enhance solubilization of brain homogenate is incubated at RT overnight and diluted the following day in 1:10 in the Biosource diluent with protease inhibitor cocktail, and centrifuged at 14,000 rpm for 20 min at 4° C. and the supernatant used for ELISA assays. To confirm detection of Aβ peptides and APP proteins we use Western blot and immunoprecipitation procedures above.

Analysis of metals in brain. Atomic absorption spectrophotometer measurements of weighed brain tissue samples are run as above. To confirm measurements, colorimetric Quantichrom kits are used. Metal levels are expressed as μg/g tissue (dry weight). Timm stain histochemistry and Rhodanine methods are run at Excalibur Pathology to determine if Cu can be localized in brain with these methods in some histological sections prepared above.

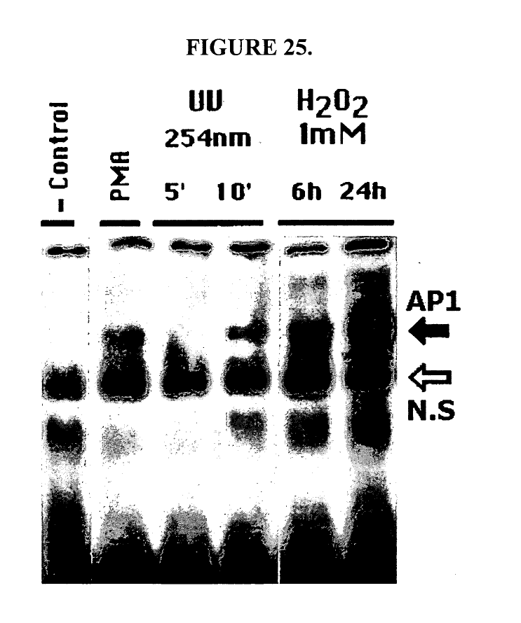

Analysis of oxidative stress. H₂O₂ oxidative stress is implicated in Aβ-Cu complex action, and is a well-characterized cataractogenic agent. Evidence described above links Cu with increased APP expression, and we demonstrated APP and Aβ increases in response to oxidative stress in cultured monkey and rat lenses exposed to H₂O₂ or UV stress (FIG. 24). To date, consistent biomarkers for cataract or lens oxidative stress have not been defined. However, in our study we also demonstrated concomitant with increased APP and Aβ, with activation of stress-associated AP-1 transcription factor in electrophoretic mobility shift assays (EMSA) in the same lens extracts (FIG. 25).

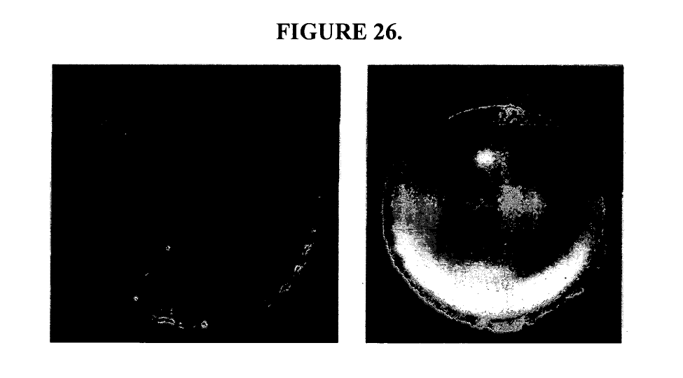

Optimizing sensitivity for Raman. Increased Aβ production in cultured lenses has been described previously by our lab and lens opacificatioin is also demonstrated in FIG. 26 by addition of a bolus of 250 μm H₂O₂ upon further incubation: Using this procedure Aβ peptides and Cu in these lenses are measured as above. Preliminary analysis indicates that Aβ peptide increases are accompanied by increased Cu accumulation. Those data also agree cataract produced with a high intensity YAG laser that detected of >2× increased Cu and unchanged Zn in rabbit lenses. In previous in vitro studies Laser probes at 514 nm (82), and 647 nm (34) wavelengths were used for detection and characterization of Aβ-metal complex signature intensities in situ in lenses.

Tissue specimens Human donor lenses are obtained from the National Disease Registry Interexchange, Philadelphia Pa. and from Vision Share, Apex NC (letters in appendix). Groups include normal controls, cataractous lenses, and lenses from confirmed AD cases. Lens specimens in groups will be collected from age <20, 20-40, 40-60, and 60-80+ years-old. Vision Share works with a consortium of Eye Banks across the country to facilitate locating specific donor tissue. As in the past, both eye globes are received intact from the same donor. Lenses are removed and classified according to the nature and quality of lens opacification using procedures outlined in the Cooperative Cataract Research Group Lens Criteria (CCRG). Lenses are photographed in dark-background, and additionally using slit-lamp photomicroscopy. It has been generally accepted in the many studies using eye bank material, that time after death does not significantly affect lenses, which are sealed and protected from the aerobic environment.

It is understood that the examples and embodiments described herein are for illustrative purposes only and that various modifications or changes in light thereof will be suggested to persons skilled in the art and are included within the spirit and purview of this application and are considered within the scope of the appended claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims.

1. A method of diagnosing an amyloid-related disorder or a predisposition thereto in a subject comprising the steps of detecting a metal-protein complex in at least one of a nuclear, supranuclear, peripheral or cortical region of an ocular lens, wherein said metal-protein complex comprises at least one amyloid protein selected from the group consisting of β-amyloid precursor protein (APP), β-amyloid protein (Aβ), Aβ₁₋₄₀, and Aβ₁₋₄₂, and wherein an increase in the amount of metal-protein complex compared to a normal control value indicates that the subject is suffering from or is at risk of developing an amyloid-related disorder.

2. The method of claim 1, wherein the metal-protein complex is detected by a Raman spectroscopy technique.

3. The method of claim 3, wherein the amyloid-related disorder is at least one member selected from the group consisting of Alzheimer's Disease (AD), Familial AD, Sporadic AD, and cataracts.

4. The method of claim 1, wherein the metal-protein complex is detected in a supranuclear region of the lens.

5. The method of claim 1, wherein the metal-protein complex is detected in a peripheral region of the cortex or supranuclear region of the lens.

6. The method of claim 1, wherein the amyloid-related disorder is Alzheimer's Disease.

7. The method of claim 1, wherein the amyloid protein is β-amyloid precursor protein (APP) or a fragment thereof.

8. The method of claim 1, wherein the amyloid protein is Aβ or a fragment thereof.

9. The method of claim 1, wherein the amyloid protein is Aβ₁₋₄₂.

10. The method of claim 1, wherein the metal-protein complex further comprises an ocular crystallin protein.

11. The method of claim 10, wherein the crystallin protein is at least one member selected from the group consisting of an α-crystallin, β-crystallin, and γ-crystallin.

12. The method of claim 1, wherein the metal-protein complex is localized in a cytosol of an lens cortical fiber cell.

13. A method of diagnosing an amyloid-related disorder or a predisposition thereto in a mammal, comprising illuminating mammalian lens tissue with an excitation light beam and detecting scattered light emitted from said tissue, wherein an increase in scattered light emitted from at least one of a nuclear, supranuclear, peripheral or cortical region of an ocular lens is indicative of the presence of a metal-protein complex, wherein the metal-protein complex comprises at least one amyloid protein selected from the group consisting of β-amyloid precursor protein (APP), Aβ, and Aβ₁₋₄₂, and wherein the increase indicates that the mammal is suffering from or is at risk of developing an amyloid-related disorder.

14. The method of claim 13, wherein the method further comprises comparing an amount of scattered light from a nuclear region of the lens tissue, wherein an increase in the ratio of supranuclear or cortical scattering to nuclear scattering indicates that the mammal is suffering from or is at risk of developing an amyloid-related disorder.

15. The method of claim 14, wherein the amyloid-related disorder is selected from the group consisting of Alzheimer's Disease (AD), Familial AD, Sporadic AD, and cataracts.

16. The method of claim 13, wherein the amyloid-related disorder is Alzheimer's Disease.

17. The method of claim 13, wherein the excitation light beam is a low wattage laser light.

18. The method of claim 13, wherein the scattered light is detected by a Raman spectroscopic technique.

19. The method of claim 18, wherein the Raman spectra yield at least one peak at about 1604 cm⁻¹, 1586 cm⁻¹ or a combination of both, indicating the presence of an Aβ-metal complex.

20. A method of diagnosing an amyloid-related cataract or a predisposition thereto in an ocular lens of a subject comprising the steps of detecting a metal-protein, complex in at least one of a nuclear, supranuclear, peripheral or cortical region of the ocular lens using Raman Spectroscopy, wherein said metal-protein complex comprises at least one amyloid protein selected from the group consisting of β-amyloid precursor protein (APP), β-amyloid protein (Aβ), Aβ₁₋₄₀, and Aβ₁₋₄₂, indicated by at least one peakat about 1604 cm⁻¹, 1586 cm⁻¹ or a combination of both, and wherein an increase in the amount of metal-protein complex compared to a normal control value indicates that the subject is suffering from or is at risk of developing an amyloid-related cataract or AD.
